(19) United States
(12) Patent Application Publication
(10) Pub. No.: US 2020/0132675 A1
(43) Pub. Date: Apr. 30, 2020

(54) MULTIPLEXED INSTRUMENT-FREE BAR-CHART SPINCHIP INTEGRATED WITH NANOPARTICLE-MEDIATED APTASENSORS FOR VISUAL QUANTITATIVE DETECTION OF MULTIPLE PATHOGENS

(71) Applicant: Board of Regents, The University of Texas System, Austin, TX (US)

(72) Inventors: XiuJun Li, El Paso, TX (US); Delfina Dominguez, El Paso, TX (US); Xiafeng Wei, El Paso, TX (US)

(21) Appl. No.: 16/514,521

(22) Filed: Jul. 17, 2019

Related U.S. Application Data
(60) Provisional application No. 62/699,525, filed on Jul. 17, 2018.

(51) Int. Cl. G01N 33/50; G01N 33/558; B01L 3/00; G01N 33/569

(52) U.S. Cl. CPC G01N 33/5091; G01N 33/558; B01L 2200/10; G01N 33/56911; B01L 3/50273

(57) ABSTRACT

A point-of-care testing (POCT) quantitative pathogen detection device is provided, without the aid of any detectors. In an illustrative embodiment, a POCT pathogen detection device includes an inlet microwell for receiving a substance, the inlet microwell connected to a distribution channel to distribute the substance to an analyzing component. The device also includes an analyzing component. that includes a first pathogen detection component. The first pathogen detection component includes a platinum nanoparticle-labeled magnetic DNA-probe configured to propel a dye through a bar-chart channel when a pathogen in the substance reacts platinum nanoparticle-labeled magnetic DNA-probe. The platinum nanoparticle-labeled magnetic DNA-probe is configured to react with a first pathogen. The device also includes at least one magnet configured to keep unreacted platinum nanoparticle-labeled magnetic DNA-probe in a sample recognition microwell, thereby inhibiting propulsion of the dye into the bar-chart channel when the pathogen is not detected.

### CROSS-REFERENCE TO RELATED CASE(S)

This application claims the benefit of U.S. Provisional Application Ser. No. 62/699,525, filed Jul. 17, 2018, entitled “Multiplexed Instrument-Free Bar-Chart Spinchip Integrated with Nanoparticle-Mediated Aptasensors for Visual Quantitative Detection Of Multiple Pathogens”, which is incorporated herein by reference in its entirety.

### BACKGROUND INFORMATION

### 1. Field

The present disclosure relates to devices, systems, and methods for detecting the presence of multiple types of pathogens in a sample material.

### 2. Background

Point-of-care testing (POCT) is medical diagnostic testing at or near the point of care, e.g., at the time and place of patient care. This contrasts with the historical pattern in which testing was wholly or mostly confined to the medical laboratory, which entailed sending specimens away from the point of care and then waiting hours or days to learn the results, during which time care must continue without the desired information.

### SUMMARY

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

In an illustrative embodiment, a point-of-care testing (POCT) pathogen detection device includes an inlet microwell for receiving a substance, the inlet microwell connected to a distribution channel to distribute the substance to an analyzing component. The device also includes an analyzing component. The analyzing component includes a first pathogen detection component. The first pathogen detection component includes a platinum nanoparticle-labeled magnetic DNA-probe configured to propel a dye through a bar-chart channel when a pathogen in the substance reacts with platinum nanoparticle-labeled magnetic DNA-probe. The platinum nanoparticle-labeled magnetic DNA-probe is configured to react with a first pathogen. The device also includes at least one magnet configured to keep unreacted platinum nanoparticle-labeled magnetic DNA-probe in an amplification microwell, thereby inhibiting propulsion of the dye into the bar-chart channel in the absence of a pathogen.

In an illustrative embodiment, a multiplexed bar-chart spinchip for instrument free visual quantitative detection of multiple pathogens for point-of-care testing (POCT) includes a layer-one sheet, a layer-two sheet, a layer-three sheet, a layer-four sheet, a layer-five sheet, and a plurality of magnets. The layer-one sheet includes a layer-one first surface and a layer-one second surface. The layer-one sheet further includes a layer-one inlet connected to a plurality of layer-one branched channels and a plurality of layer-one exhaust outlets. The layer-two sheet includes a layer-two first surface and a layer-two second surface. The layer-two first surface is in contact with the layer-one second surface. The layer-two sheet further includes a plurality of layer-two sample inlets, a plurality of layer-two substrate inlets, a plurality of layer-two indicator inlets, a plurality of layer-two exhaust outlets, and a plurality of layer-two outlets. The layer-three sheet includes a layer-three first surface and a layer-three second surface. The layer-three first surface is bonded to the layer-two second surface. The first surface of layer-three sheet further includes a plurality of sample recognition microwells, a plurality of catalytic amplification microwells, a plurality of indicator microwells, a plurality of “T”-phase exchange channels, a plurality of connection channels, and a plurality of bar-chart channels. Each of the sample recognition microwells comprise a platinum nanoparticle-labeled magnetic DNA-probe. The platinum nanoparticle-labeled magnetic DNA-probe in one of the sample recognition microwells is different from at least one of the other platinum nanoparticle-labeled magnetic DNA-probes in another one of the sample recognition microwells. Each of the sample recognition microwells is connected with a corresponding one of the catalytic amplification microwells and a corresponding one of the indicator microwells by a corresponding one of the “T”-phase exchange channels. Besides, each of the connection channels is configured to specially connect a corresponding one of the sample recognition microwells and a corresponding one of the catalytic amplification microwells. The plurality of sample recognition microwells, the plurality of catalytic amplification microwells, the plurality of indicator microwells, the plurality of “T”-phase exchange channels, the plurality of connection channels, and the plurality of bar-chart channels form a plurality of parallel microfluidic units. The layer-four sheet includes a layer-four first surface and a layer-four second surface. The layer-four first surface contacts the layer-three second surface. The layer-four second surface includes a plurality of hollow microwells. Each of the magnets is residing in one of the hollow microwells. The layer-five sheet includes a layer-five first surface and a layer-five second surface. The layer-five first surface contacts the layer-four second surface, thereby securing the magnets in the hollow microwells. The magnets keep unreacted platinum nanoparticle-labeled magnetic DNA-probe in a corresponding sample recognition microwells. A dye is forced through one of the bar-chart channels when a sample is introduced with a pathogen corresponding to the particular platinum nanoparticle-labeled magnetic DNA-probe, corresponding to the particular bar-chart channels. In an illustrative embodiment, a distance that the dye moves is proportional to an analyte concentration, thus enabling quantitative detection of pathogens, without the aid of any detectors.

In an illustrative embodiment, a point-of-care testing (POCT) device for quantitative pathogen detection includes an inlet microwell for receiving a substance. The POCT device also includes an analyzing component configured to propel a dye through a bar-chart channel, when a pathogen in the substance reacts with a catalyst nanoparticle-labeled magnetic DNA-probe. The POCT device also includes at least one magnet, configured to keep unreacted catalyst nanoparticle-mediated magnetic DNA-probe in an amplification microwell, thereby inhibiting propulsion of the dye into the bar-chart channel, when the pathogen is not detected. The inlet microwell is connected to a distribution channel to distribute the substance to the analyzing component.

In an illustrative embodiment, a method of fabricating a point-of-care testing (POCT) device for quantitative pathogen detection includes forming an inlet microwell for receiving a substance, the inlet microwell connected to a distribution channel to distribute the substance to an analyzing component. The method also includes forming an analyzing component comprising a first pathogen detection component. The first pathogen detection component includes a platinum nanoparticle-labeled magnetic DNA-probe configured to recognize the first pathogen and then generate a fragmentary DNA-platinum nanoparticles. The fragmentary DNA-platinum nanoparticles are configured to react with the substrate to generate gas to propel a dye through a bar-chart channel when a pathogen in the substance reacts with platinum nanoparticle-labeled magnetic DNA-probe. The platinum nanoparticle-labeled magnetic DNA-probe is configured to react with a first pathogen. The method also includes forming a layer, that includes at least one magnet configured to keep unreacted platinum nanoparticle-labeled magnetic DNA-probe in an amplification microwell, thereby inhibiting propulsion of the dye into the bar-chart channel when the pathogen is not detected.

### BRIEF DESCRIPTION OF THE DRAWINGS

The accompanying figures, in which like reference numerals refer to identical or functionally-similar elements throughout the separate views and which are incorporated in and form a part of the specification, further illustrate the embodiments and, together with the detailed description, serve to explain the principles of the disclosed embodiments.

FIG. 1 is a block diagram of a Multiplexed Bar-chart SpinChip (MB SpinChip) integrated with nanoparticle-mediated magnetic aptasensors in accordance with an illustrative embodiment;

FIG. 2A is a diagram of an exploded view of an exemplary embodiment of an MB-SpinChip with 5 patterned PMMA sheets in accordance with an illustrative embodiment;

FIG. 2B is a photograph of an exemplary embodiment of an assembled MB-SpinChip in accordance with an illustrative embodiment;

FIG. 2C is a 3D schematic of an exemplary embodiment of an assembled MB-SpinChip in accordance with an illustrative embodiment;

FIG. 3 shows a schematic of an assay procedure in accordance with an illustrative embodiment;

FIGS. 4A and 4B show a photograph and corresponding histogram of visual bar-chart at different conditions in accordance with an illustrative embodiment;

FIGS. 5A-5C show optimization of (A) DNA probe washing times, (B) concentration ratio of beads-DNA and PtNPs-aptamer, and (C) the reaction time between DNA probes and pathogens in accordance with an illustrative embodiment;

FIGS. 6A-6B show selectivity investigation of an exemplary embodiment of the MB-SpinChip with different DNA probes for S. enterica, E. coli, and L. monocytogenes (abbreviated as L. mono) by their corresponding photographs (A) and bar-length histogram (B) in accordance with an illustrative embodiment;

FIGS. 7A-7C show a visual quantitative pathogen detection using the MB-SpinChip in accordance with an illustrative embodiment;

FIGS. 8A-8E show a process of multiplexed pathogen detection in accordance with an illustrative embodiment;

FIG. 9 shows an example of multiplexed visual quantification in accordance with an illustrative embodiment;

FIG. 10 shows a TEM photograph of PtNPs in accordance with an illustrative embodiment;

FIGS. 11A-11F show schematic diagrams of various layers of an MB-SpinChip in accordance with an illustrative embodiment;

FIGS. 12A-12D show operation steps of the MB-SpinChip in accordance with an illustrative embodiment;

FIGS. 13A-13C show selectivity investigation of the MB-SpinChip with different DNA probes for (A) S. enterica, (B) E. coli, and (C) L. monocytogenes in accordance with an illustrative embodiment; and

FIG. 14 shows a response of an exemplary embodiment of a MB-SpinChip operated by multiple users to 20 pM PtNPs and 30% H2O2 in 5 minutes in accordance with an illustrative embodiment.

### DETAILED DESCRIPTION

The particular values and configurations discussed in these non-limiting examples can be varied and are cited merely to illustrate at least one embodiment and are not intended to limit the scope thereof.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer-readable program instructions.

The different illustrative examples recognize and take into account one or more different considerations. For example, the illustrative examples recognize and take into account that a pathogen detection device for food safety is desirable. The illustrative embodiments recognize and take into account that in many developing countries, detecting pathogens in food is difficult and many places do not have access to expensive high quality equipment to determine if a food product is contaminated. The illustrative embodiments recognize and take into account that it would be desirable to have a low cost and simple device and method to ascertain whether a food product is contaminated.

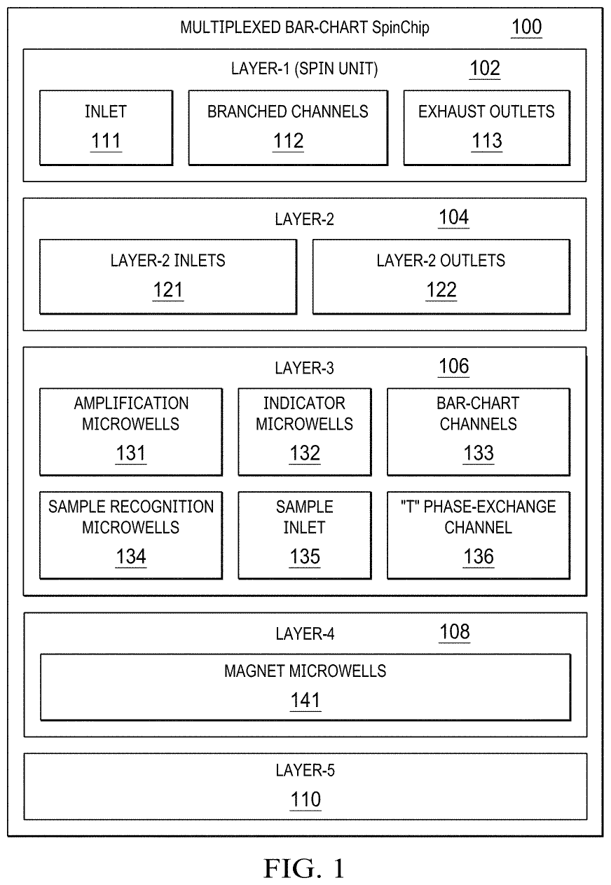

FIG. 1 is a block diagram of an exemplary embodiment of a Multiplexed Bar-chart SpinChip (MB SpinChip) 100 integrated with nanoparticle-mediated magnetic aptasensors. The MB SpinChip 100 includes a layer-1 102, a layer-2 104, a layer-3 106, a layer-4 108, and a layer-5 110. Layer-1 102 is a spin unit. Layer-1 102 includes an inlet 111, a plurality of branched channels 112, and one or more exhaust outlets 113. Layer-2 104 includes layer-2 inlets 121 and layer-2 outlets 122. Layer-3 106 includes amplification microwells 131, indicator microwells 132, bar-chart channels 133, sample recognition microwells 134, a sample inlet 135, and a “T” phase-exchange channel 136. Layer-4 108 includes a magnet microwell 141. Layer-5 110 covers the magnet microwells 141 to hold the magnets secure in each of the magnet microwells 141. The layers 102, 104, 106, 108, 110 may be fabricated from, for example, poly(methyl methacrylate) (PMMA) (also known as acrylic or acrylic glass) or from glass. PMMA is cheaper than glass and in some embodiments, better than glass. The spin unit not only allows for reagent introduction, but also solves a cross interference problem between the various analyzing components that each analyze the substance for the presence of a different pathogen.

In an illustrative embodiment, a point-of-care testing (POCT) pathogen detection device includes an inlet microwell for receiving a substance, the inlet microwell connected to a distribution channel to distribute the substance to an analyzing component. The device also includes an analyzing component. The analyzing component includes a first pathogen detection component. The first pathogen detection component includes a platinum nanoparticle-labeled magnetic DNA-probe, configured to propel a dye through a bar-chart channel, when a pathogen in the substance reacts with the platinum nanoparticle-labeled magnetic DNA-probe. The platinum nanoparticle-labeled magnetic DNA-probe is configured to react with a first pathogen. The device also includes at least one magnet configured to keep unreacted platinum nanoparticle-labeled magnetic DNA-probe in an amplification microwell, thereby inhibiting propulsion of the dye into the bar-chart channel when the pathogen is not detected.

In an illustrative embodiment, the distribution channel comprises a plurality of distribution channels and the analyzing component includes a plurality of pathogen detection components. Each of the pathogen detection component is configured to detect a different pathogen. Each of the pathogen detection components includes a platinum nanoparticle-labeled magnetic DNA-probe, configured to propel a dye through a bar-chart channel, when a pathogen in the substance reacts with the platinum nanoparticle-labeled magnetic DNA-probe. Each of the platinum nanoparticle-labeled magnetic DNA-probes is configured to react with a different pathogen.

In an illustrative embodiment, the at least one magnet includes a magnet for each of the plurality of analyzing components.

In an illustrative embodiment, the platinum nanoparticle-labeled magnetic DNA-probe includes an aptamer-DNA-platinum nanoparticle.

In an illustrative embodiment, the device includes a spin-chip.

In an illustrative embodiment, each of the plurality of pathogen detection components is configured to receive a portion of a single sample, received at the inlet microcell.

In an illustrative embodiment, each of the plurality of pathogen detection components is isolated from the other ones of the plurality of pathogen detection components by rotating the Spin unit to disconnect the inlet from other bar-chart channels such that a reaction to a pathogen in a first pathogen detection component does not propel a dye in a second pathogen detection component into the bar-chart channel corresponding to the second pathogen detection component.

In an illustrative embodiment, the platinum nanoparticle-labeled magnetic DNA-probe includes a DNA hybridization between magnetic capture-DNA-beads and aptamer-DNA-platinum nanoparticles.

In an illustrative embodiment, a multiplexed bar-chart spinchip for instrument-free visual quantitative detection of multiple pathogens for point-of-care testing (POCT) includes a layer-one sheet, a layer-two sheet, a layer-three sheet, a layer-four sheet, a layer-five sheet, and a plurality of magnets. The layer-one sheet includes a layer-one first surface and a layer-one second surface. The layer-one sheet further includes a layer-one inlet connected to a plurality of layer-one branched channels and a plurality of layer-one exhaust outlets. The layer-two sheet includes a layer-two first surface and a layer-two second surface. The layer-two first surface is in contact with the layer-one second surface. The layer-two sheet further includes a plurality of layer-two sample inlets, a plurality of layer-two substrate inlets, a plurality of layer-two indicator inlets, a plurality of layer-two exhaust outlets, and a plurality of layer-two outlets. The layer-three sheet includes a layer-three first surface and a layer-three second surface. The layer-three first surface is bonded to the layer-two second surface. The first surface of layer-three sheet further includes a plurality of sample recognition microwells, a plurality of catalytic amplification microwells, a plurality of indicator microwells, a plurality of “T”-phase exchange channels, a plurality of connection channels, and a plurality of bar-chart channels. Each of the sample recognition microwells comprise a platinum nanoparticle-labeled magnetic DNA-probe. The platinum nanoparticle-labeled magnetic DNA-probe in one of the sample recognition microwells is different from at least one of the other platinum nanoparticle-labeled magnetic DNA-probes in another one of the sample recognition microwells. Each of the sample recognition microwells is connected with a corresponding one of the catalytic amplification microwells and a corresponding one of the indicator microwells by a corresponding one of the “T”-phase exchange channels. Besides, each of the connection channels is configured to specially connect a corresponding one of the sample recognition microwells and a corresponding one of the catalytic amplification microwells. The plurality of sample recognition microwells, the plurality of catalytic amplification microwells, the plurality of indicator microwells, the plurality of “T”-phase exchange channels, the plurality of connection channels, and the plurality of bar-chart channels form a plurality of parallel microfluidic units. The layer-four sheet includes a layer-four first surface and a layer-four second surface. The layer-four first surface contacts the layer-three second surface. The layer-four second surface includes a plurality of hollow microwells. Each of the magnets is residing in one of the hollow microwells. The layer-five sheet includes a layer-five first surface and a layer-five second surface. The layer-five first surface contacts the layer-four second surface thereby securing the magnets in the hollow microwells. The magnets keep unreacted platinum nanoparticle-labeled magnetic DNA-probe in a corresponding sample recognition microwell. A dye is forced through one of the bar-chart channels when a sample is introduced with a pathogen corresponding to the particular platinum nanoparticle-labeled magnetic DNA-probe corresponding to the particular bar-chart channels. In an illustrative embodiment, multiple different types of pathogens are quantitatively detected simultaneously from a single assay.

In an illustrative embodiment, the platinum nanoparticle-labeled magnetic DNA-probes each include a DNA hybridization between magnetic beads-complementary DNA and aptamer DNA-platinum nanoparticles.

In an illustrative embodiment, a point-of-care testing (POCT) device for quantitative pathogen detection includes an inlet microwell for receiving a substance. The POCT device also includes an analyzing component configured to propel a dye through a bar-chart channel when a pathogen in the substance reacts with a catalyst nanoparticle-labeled magnetic DNA-probe (also referred to as a catalyst-mediated magnetic DNA-probe). The POCT device also includes at least one magnet configured to keep unreacted catalyst nanoparticle-mediated magnetic DNA-probe in an amplification microwell, thereby inhibiting propulsion of the dye into the bar-chart channel when the pathogen is not detected. The inlet microwell is connected to a distribution channel to distribute the substance to the analyzing component.

In an illustrative embodiment, the catalyst nanoparticle-mediated magnetic DNA-probe is configured to recognize the pathogen and generate fragmentary DNA-catalyst nanoparticles.

In an illustrative embodiment, the fragmentary DNA-catalyst nanoparticles are configured to react with the substrate to generate gas to propel a dye through a bar-chart channel when a pathogen in the substance reacts with the catalyst nanoparticle-labeled magnetic DNA-probe.

In an illustrative embodiment, the catalyst nanoparticle-labeled magnetic DNA-probe is configured to react with a first pathogen and the catalyst nanoparticle-labeled magnetic DNA-probe is configured to not react with a second pathogen. In an illustrative embodiment, the catalyst includes platinum.

In an illustrative embodiment, a method of fabricating a point-of-care testing (POCT) device for quantitative pathogen detection includes forming an inlet microwell for receiving a substance, the inlet microwell connected to a distribution channel to distribute the substance to an analyzing component. The method also includes forming an analyzing component comprising a first pathogen detection component. The first pathogen detection component includes a platinum nanoparticle-labeled magnetic DNA-probe configured to recognize the first pathogen and then generate a fragmentary DNA-platinum nanoparticles. The fragmentary DNA-platinum nanoparticles are configured to react with the substrate to generate gas to propel a dye through a bar-chart channel when a pathogen in the substance reacts with platinum nanoparticle-labeled magnetic DNA-probe. The platinum nanoparticle-labeled magnetic DNA-probe is configured to react with a first pathogen. The method also includes forming a layer that includes at least one magnet configured to keep unreacted platinum nanoparticle-labeled magnetic DNA-probe in an amplification microwell, thereby inhibiting propulsion of the dye into the bar-chart channel when the pathogen is not detected.

Although described herein primarily with reference to platinum nanoparticle-labeled magnetic DNA-probes, other magnetically susceptible materials other than platinum may be used. For example, in other embodiments, the nanoparticle-labeled magnetic DNA-probes are gold nanoparticle-labeled magnetic DNA-probes, prussian-blue nanoparticle-labeled magnetic DNA-probes, ferrocobalt nanoparticle-labeled magnetic DNA-probes, and/or catalase-labeled magnetic DNA-probes. More details about exemplary embodiments of MB-SpinChips are described below.

A portable Multiplexed Bar-chart SpinChip (MB-SpinChip) integrated with nanoparticle-mediated magnetic aptasensors was developed for visual quantitative instrument-free detection of multiple pathogens. This versatile multiplexed SpinChip combines aptamer specific recognition and nanoparticle-catalyzed pressure amplification to achieve a sample-to-answer output for sensitive point-of-care testing (POCT). This is the first report of pathogen detection using a volumetric bar-chart chip and it is also the first bar-chart chip using a “Spinning” mechanism to achieve multiplexed bar-chart detection. Additionally, the introduction of the Spin unit not only enabled convenient sample introduction from one inlet to multiple separate channels in the multiplexed detection, but also elegantly solved the pressure cross interference problem in the multiplexed volumetric bar-chart chip. This user-friendly MB-SpinChip allows visual quantitative detection of multiple pathogens simultaneously with high sensitivity but without utilizing any specialized instruments. Using this MB-SpinChip, three major foodborne pathogens including Salmonella enterica (S. enterica), Escherichia coli (E. coli), and Listeria monocytogenes (L. monocytogenes) were specifically quantified in apple juice with limits of detection of about 10 CFU/mL. This MB-SpinChip with a bar-chart-based visual quantitative readout has great potential for the rapid simultaneous detection of various pathogens at the point-of-care and wide applications in food safety, environmental surveillance, and infectious disease diagnosis.

Numerous laboratory detection techniques have been developed and standardized for various applications such as food safety surveillance and diagnosis of infectious diseases caused by pathogens. For instance, as reported by the World Health Organization (WHO), each year almost 1 in 10 people (estimated 600 million globally) get ill after orally taking unsafe food and 420,000 die in the world.¹ To monitor food safety and infectious diseases, multiple instrumental analysis methods including fluorescence,²⁻⁴ electrochemistry, colorimetry, surface enhanced Raman scattering, and chromatography¹³ have been developed for pathogen identification and quantification. However, those methods require costly and cumbersome instruments, moderate laboratory conditions, sophisticated operations, and well-trained professional personnel. Those factors become major roadblocks for these conventional methods to be employed to provide timely monitoring of pathogens on site and in low-resource settings such as developing nations. As per the ASSURED criteria from WHO,¹⁴ the point-of-care testing (POCT) should be advocated to be affordable, sensitive, specific, user-friendly, rapid & robust, equipment-free, and deliverable to end users especially in the developing countries or resource-limited regions. Therefore, the development of cost-effective, user-friendly, and quantitative POC methods is in great need.

Over past decades, considerable microfluidic POCTs have been employed to meet the challenges and requirements. Firstly, some handheld-devices or cellphone-assistant platforms were built to achieve the low-cost portable detection. Several photothermal, colorimetric, glucose-metric, pressuremetric, centrifuge-based and camera-based systems were proposed to displace expensive instruments with frequently-used portable devices, such as a thermometer, cellphone, glucometer, and barometer, etc. For example, one group developed a novel photothermal biomolecular quantitation method using a common thermometer as the quantitative signal reader. The nanoparticle-mediated photothermal effect was first introduced in immunoassays for quantitation of various disease biomarkers and proteins, achieving a low-cost, portable, and quantitative readout method for nonprofessional people. Although reducing the cost in instrumentation with those frequently-used portable detectors, low-degree integration and accessary readout detectors still limit the development and application of related methods in remote regions. Real equipment-free setup requires neither an excitation source, such as light or electricity, nor an additional signal detector, which are hard to be concurrently fulfilled in POCTs. Secondly, due to the low-cost nature of paper, a hot research field was focused on paper-based platforms to develop series of instrument-free analytical methods, such as colorimetric, timebased, and counter-based paper-based microfluidic devices. Many features including reagent storage, filtration, reaction incubation, and capillary driving have been integrated on paper-based microfluidic devices. However, concessive sensitivity and low-throughput restrict the paper-based POCTs' generality and detection sensitivity. For instance, colorimetric detection offers an attractive visual detection approach for POC detection on low-cost paper-based microfluidic devices. Nevertheless, the sensitivity is low, and it is challenging for colorimetric detection to achieve quantitative analysis without the aid of other advanced equipment, reaching a bottleneck for the paper-based colorimetric assay to be widely used in practice. Thirdly, microfluidic volumetric bar-chart chips were designed as a high-degree integrative platform for visual quantitative detection based on the distance, where a color dye plug moves through a channel without using pneumatic pumps and signal collection devices. For example, Qin's group reported an ELISA-based competitive volumetric bar-chart chip for the on-site detection of small molecules, cancer biomarkers, and drug abuse screening, which also ingeniously achieved multiplexed detection by a slip operation. Moreover, using a “competition mode”, a real-time internal control was embedded in the POC chip to decrease the potential influence of the background resulting in few false-negative or false-positive results. However, this platform still suffers two major drawbacks: the tedious and costly fabrication of glass chips, complex operation procedures, and temperature-sensitive enzymes employed as the catalysts, limiting their applications for resource-poor settings such as on-site or field detection.

Due to excellent catalysis performance and robustness at the ambient temperature for the on-site detection, various nanomaterials have been employed as catalysts in the POCTs. Compared with traditional enzyme-based catalytic reactions, nanomaterials can provide more stable and efficient catalytic properties for signal amplification, such as higher sensitivity by versatile high-surface-to-volume-ratio nanostructures, higher robustness in a complex non-lab setting, and versatile functionalization via a controllable self-assembly or surface modification. Numerous metallic and carbon-based nanomaterials were reported as highly sensitive catalysts for colorimetric, chemiluminescent or electrochemical detection. For instance, one group reported that PtNPs generated more than 400-times O₂ per second than common catalase, resulting in much higher detection sensitivity than catalase methods. In addition, a new iron oxide-to Prussian blue (PB) nanoparticle (NP) conversion strategy was developed and applied to sensitive colorimetric immunosensing of cancer biomarkers by Fu et al. Utilizing the highly visible blue color change, this PB NPs-mediated colorimetric system can achieve a LOD of 1.0 ng/mL for the prostate specific antigen (PSA), with the LOD of about 80 folds lower than that of common AuNP-based colorimetric assays.

Herein, we developed a multiplexed bar-chart SpinChip integrated with nanomaterial-mediated aptasensors for visual quantitative instrument-free detection of multiple pathogens at the point of care, given the urgent demand for POCTs from pathogen detection and disease diagnosis. Three major foodborne pathogens, i.e. Salmonella enterica (S. enterica), Escherichia coli (E. coli), and Listeria monocytogenes (L. monocytogenes), were used as model analytes to demonstrate the method for the visual multiplexed quantitative analysis using the MB-SpinChip. These three kinds of foodborne bacteria commonly lead to a regional epidemic situation and serious emergencies, infecting about 1.2 million, 265,000, and 2,500 persons per year by Salmonella, E. coli, and Listeria in the United States, respectively. To the best of our knowledge, this is the first volumetric bar-chart chip for pathogen detection. Aptasensors can simply identify different types of pathogenic microorganisms specifically, eliminating complicated pathogen preparation steps. Nanoparticle-mediated pressure amplification utilized in the MB-SpinChip can not only amplify detection signals, but also enable the quantitative bar-chart readout from the MB-SpinChip. Additionally, on the base of our recent work in a CD-like SpinChip for multiplexed loopmediated DNA isothermal amplification (mLAMP), we developed another Spin unit on the MB-SpinChip, which not only provided convenient sample introduction from one inlet to multiple separate channels, but also gracefully solved the pressure cross interference problem in the multiplexed volumetric bar-chart chip. Thus, our microfluidic platform doesn't need any specialized instruments for fluid manipulations or photo/electro-signal capturing devices, while maintaining the capacity for visual multiplexed quantitative analysis with high sensitivity, compared to other POC devices. Due to those significant features, our versatile MB-SpinChip can readily achieve simple quantitative sample-to-answer POC sensing in a multiplexed format in resource-limited settings.

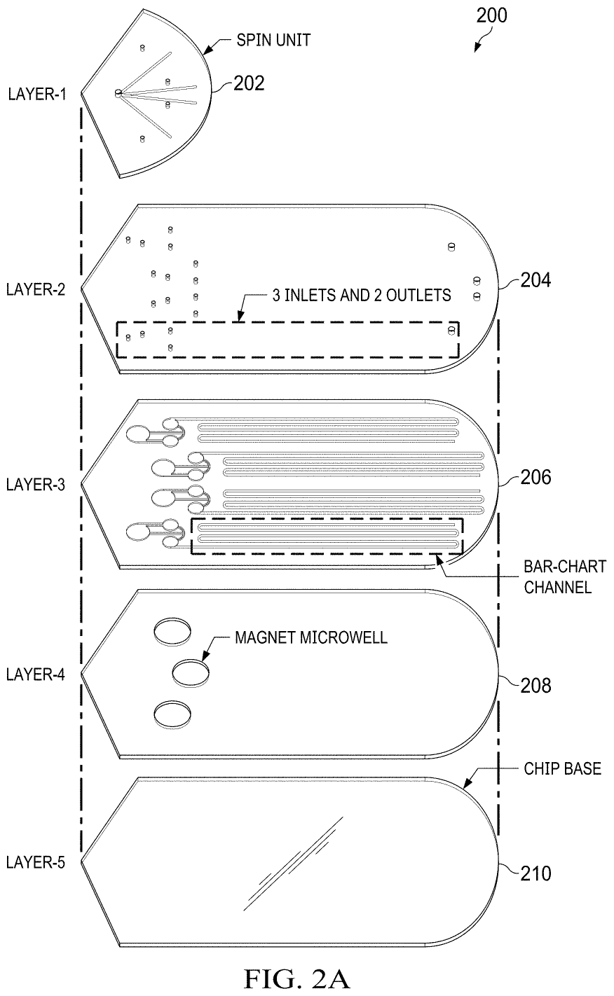

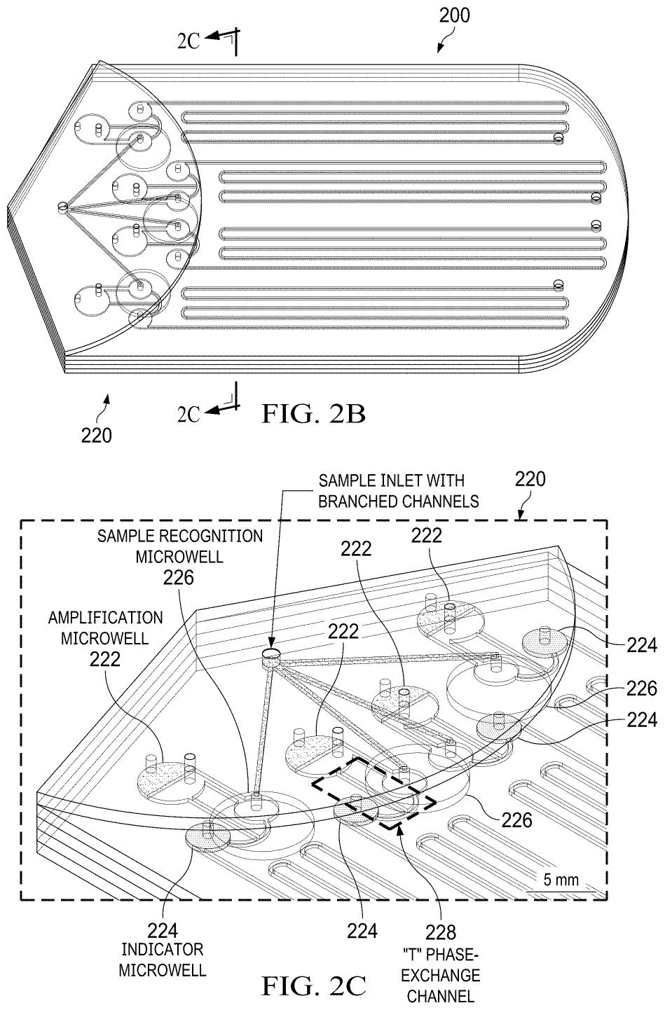

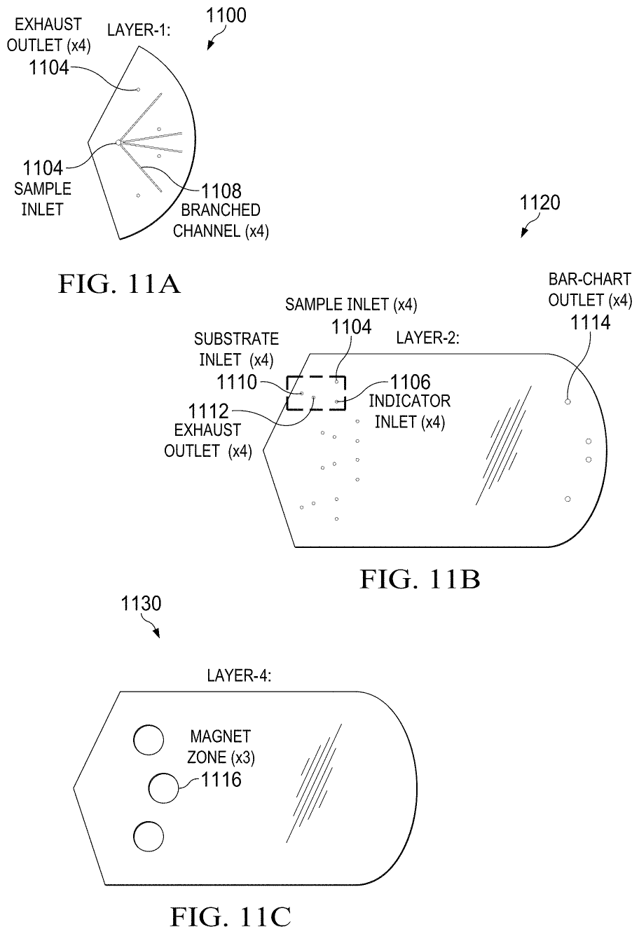

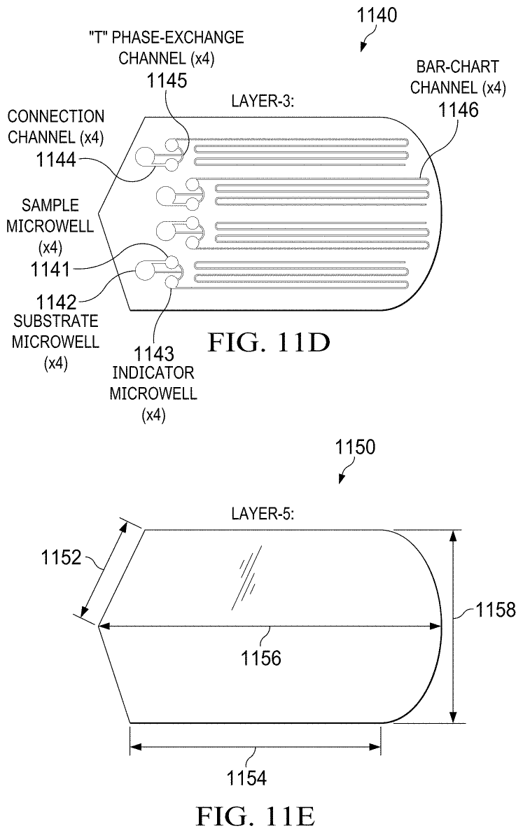

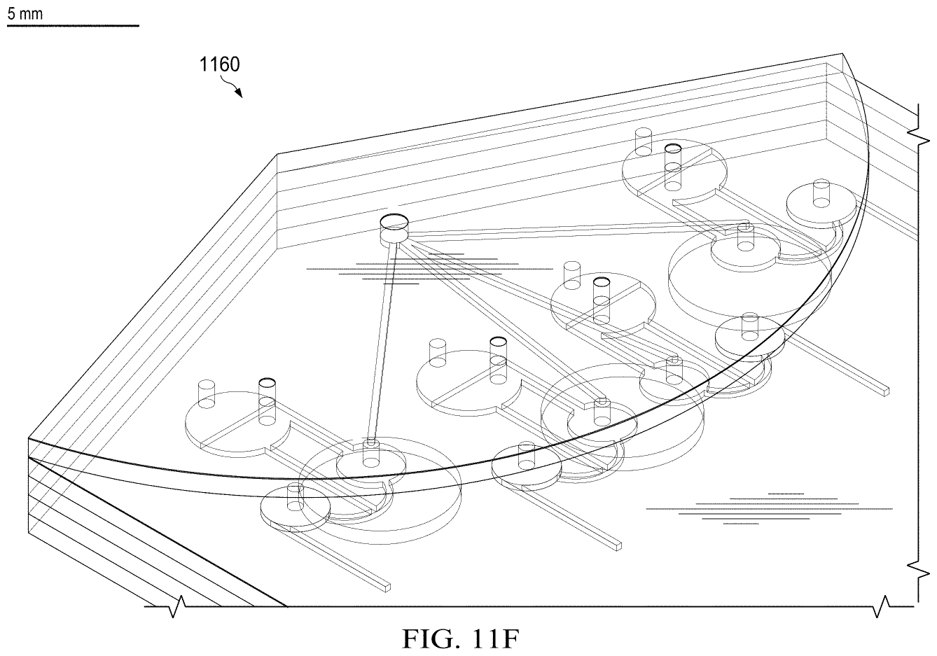

Turning now to FIGS. 2A-2C, the Layout and Fabrication of a MB-SpinChip is shown in accordance with an illustrative embodiment. In an embodiment, the pattern of each layer on the MB-SpinChip 200 was designed with Adobe AI software and ablated on 2 mm thick PMMA by a laser cutter from Epilog Laser (Golden, Colo.). As shown in FIGS. 2A-2C and 11A-11F, the MB-SpinChip 200 includes five patterned layers of PMMA. MB-SpinChip 200 may be implemented as, for example, multiplexed bar-chart SpinChip 100 in FIG. 1. Compared to FIG. 2, FIGS. 11A-11F also shows detailed specifications. In an illustrative embodiment, layer-1 sheet 202 was designed in a flabellate shape (intersection angle: ≮134°) with one common sample inlet (dark green represents a hollow hole at the diameter of 1.2 mm by the laser vector process at 60% power and 10% speed) connected with four branched channels (light green represents channels. Dimensions, 15 mm×0.3 mm; depth: 0.5 mm by the laser raster process at 40% power and 30% speed) and four exhaust outlets (0.7 mm). Layer-2 sheet 204 was laser-ablated to create three inlets and two outlets for four parallel units, including the sample inlet, the substrate inlet, the indicator inlet, the exhaust outlet, and the bar-chart channel outlet. Accordingly, Layer-3 sheet 206 includes four corresponding sets of microwells and channels for four parallel microfluidic units, including four sample recognition microwells (blue; depth, 1.5 mm by the laser raster process at 60% power and 20% speed), 4 catalytic amplification microwells (purple; depth, 1.5 mm), four indicator microwells (gold color; depth, 1.5 mm), and four bar-chart channels (depth, 0.5 mm). Each amplification microwell is connected to an indicator microwell through a connection channel (red, 6 mm×0.6 mm, 1.0 mm in depth by the laser raster process at 50% power and 25% speed). A similar “T” phase-exchange channel (width: 0.3 mm; depth: 0.5 mm) was fabricated to keep the pressure balance while connecting three microwells. Three hollow circular microwells were fabricated at the bottom surface of the Layer-4 sheet 208 to hold circle magnets. Layer-5 sheet 210 is the bottom layer to cover the magnet microwells. After the laser ablation process, the patterned Layer-2 and Layer-3 PMMA sheets were heat-bonded in an oven from VWR (Radnor, Pa.) at 150° C. for 60 min. The bonded Layer-2&3 was hydrophobicated for 30 min by fully filling the fluorinated oil which was evaporated in the air later. Afterward, 10 μL of the self-assembled DNA biosensor stock solution was injected into the sample recognition microwell and kept in the vacuum desiccator to remove the solvent. 10 μL H₂O₂ and 10 μL food dye were preinjected into the amplification microwell and the indicator microwell, respectively. The bonded Layer-2&3, Layer-4, and Layer-5 PMMA sheets were easily assembled together by using super glue. The top Layer-1 was tightened with the rest layers by a clamp, but could be manually rotated to set the Spin unit to be “ON” or “OFF”. Finally, the aptasensor integrated MB-SpinChip was stored in a plastic zipper bag at 4° C. before use.

Other experimental sections include the bacterial pathogen culture, the preparation of the DNA biosensor and the assay procedure on the MB-SpinChip are listed in the Supporting Information.

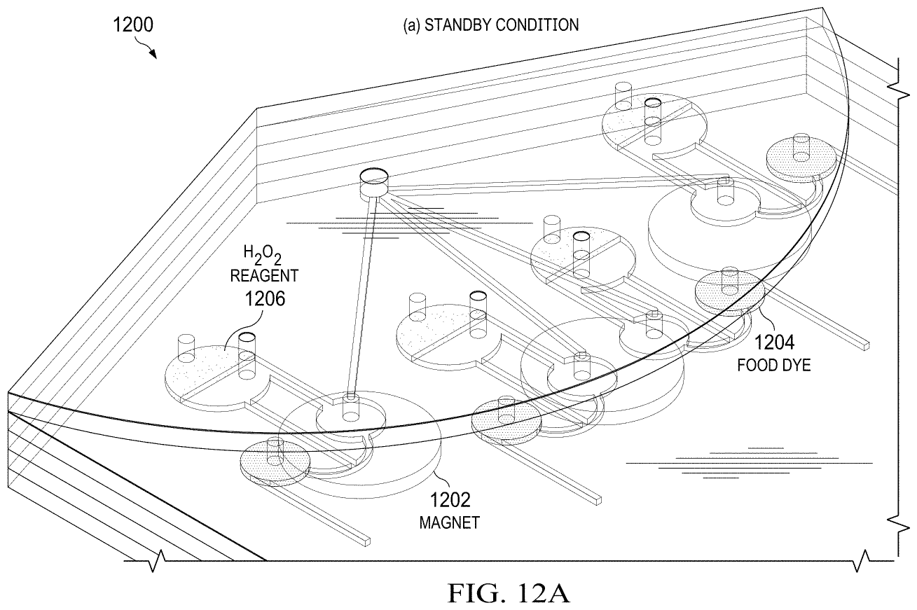

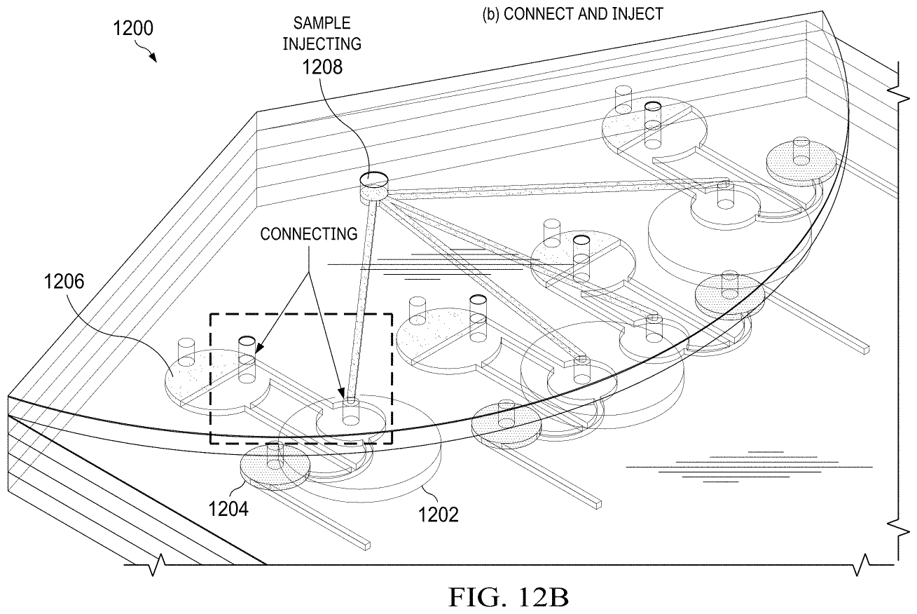

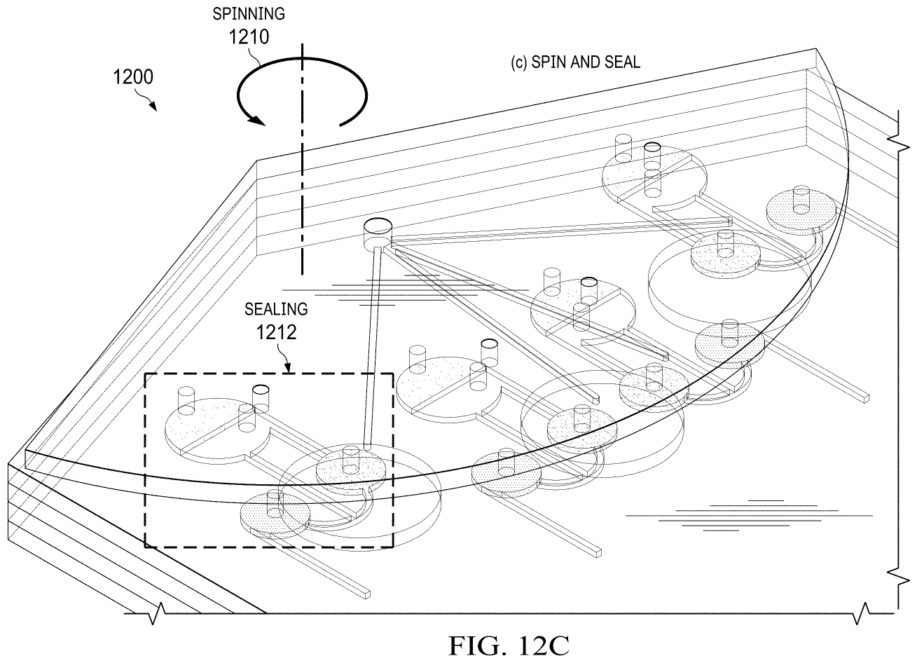

FIG. 2A shows an exploded view of an exemplary embodiment of an MB-SpinChip with 5 patterned PMMA sheets in accordance with an illustrative embodiment. FIG. 2B is a photograph of an exemplary embodiment of an assembled MB-SpinChip 200. FIG. 2C is a 3D schematic of an exemplary embodiment of a section 220 of an assembled MB-SpinChip in accordance with an illustrative embodiment. The section 220 includes a plurality of amplification microwells 222, a plurality of indicator microwells 224, a plurality of sample recognition microwells 226, and a plurality of “T” phase-exchange channels 228. For more details of the MB-SpinChip, see FIGS. 11A-11F and FIGS. 12A-12D described below.

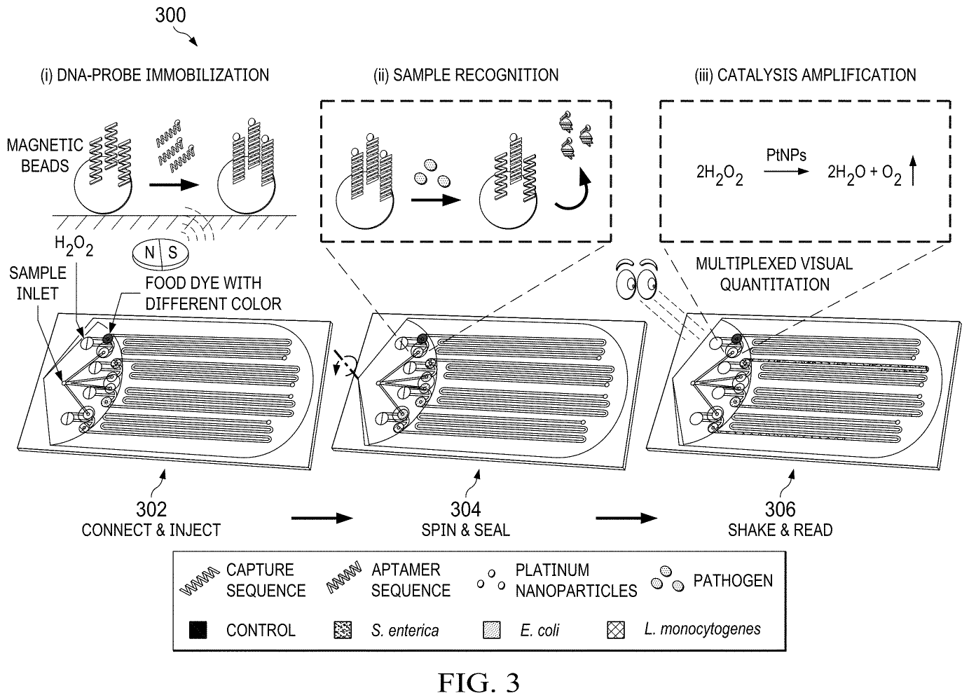

Turning now to FIG. 3, a schematic of an exemplary embodiment of the assay procedure 300 is shown. (i) DNA Probe Immobilization: H2O2 substrate solutions (light blue region), food dye solutions (yellow, dark blue, red and green circle) and DNA probes (light gray circle) are respectively pre-stored in the MB-SpinChip. Herein, magnetic DNA probes are immobilized in different sample-recognition microwells by a magnetic field (dark gray circle). (ii) Sample Recognition: pathogens specifically combine with PtNPs-aptamers to form complexes which are then released into sample solutions (purple circle). (iii) Catalysis Amplification: sample solutions and H2O2 solutions are mixed to generate O2 with the pressure increase inside, resulting in the internal pressure increase which further leads to the food dye to move into channels to form different bar-chart signals for visual multiplexed pathogen detection.

Working Principle of the MB-SpinChip for visual quantitative multiplexed detection. This MB-SpinChip is composed of four critical parts: Spin unit, sample recognition unit, catalytic amplification unit, and bar-chart unit, as shown in FIGS. 1 and 2. The pattern of each unit was carefully optimized for full functionality. The Spin unit is designed for efficient reagent delivery from one common inlet microwell to different sample recognition units or bar-chart detection units, while the sample recognition unit and catalytic amplification are designed for pathogen recognition using the aptasensor and nanomaterial mediated pressure amplification. An elaborate “T” phase exchange channel is employed for the media exchange in a sealed condition, which guarantees the smooth interchange between the sample and the air in the amplification microwell after shaking. The bar-chart unit includes dye microwells, barchart channels with scale bars to provide the visual bar-chart signal readout. FIG. 2A shows the exploded view of the MB-SpinChip 200, illustrating that three major layers (layers 1 202, 3 206 and 4 208) contain the Spin unit, major bar-chart channels and reaction wells, and magnetic beads holders, respectively. Detailed specifications are shown in FIGS. 11A-11F.

In order to measure multiple samples at a time in bar-chart microfluidics, multiple separate channels were often used for different analytes. Because those channels were independent and separate sample injections were required for different analytes, those types of multiple sample assays lacked high degree of integration, while a slight difference in sample injection can cause detection result variations after a manual slip. Using those glass-based reusable bar-chart slip-chips, several complicated operations have to be executed by trained personnel for the reagents injection, slip separation, and chip washing. In these cases, those bar-chart assays cannot be considered as genuine “sample-to-answer” by the sophisticated manual operations. Although it is not difficult for microfluidic methods to employ one inlet to introduce reagents to different locations just by adding a connection channel between a common inlet and different separate channels for reagent delivery, it will cause a pressure cross interference issue for volume bar-chart chips, because generated gas can move freely in all those connected channels. Therefore, in this work we designed a Spin unit to solve this issue, based on our recent work regarding a CD-shaped SpinChip for mLAMP. The Spin unit that we developed in this work is not only to delivery reagents, but also to disconnect each bar-chart channel by rotating the Spin unit after the sample introduction step. More detailed principle of the MB-SpinChip is discussed in the following paragraph.

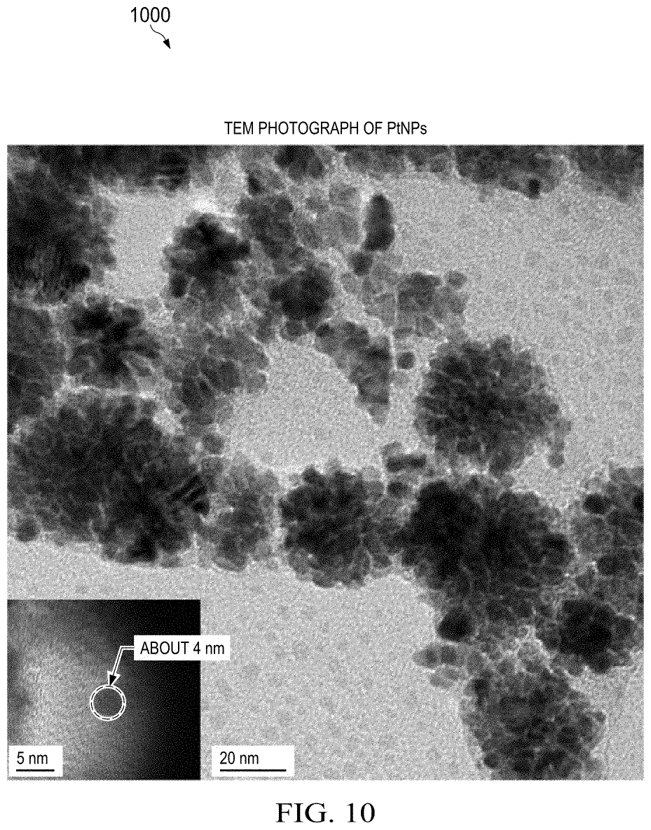

The working principle of the MB-SpinChip is composed of three main steps as illustrated in FIG. 3, including 302 Connect & Inject, 304 Spin & Seal, and 306 Shake & Read. Before sample introduction, the nanoparticle-mediated magnetic DNA-probe is assembled by DNA hybridization between magnetic capture-DNA-beads (beads-DNA) and aptamer-DNA-platinum nanoparticles (aptamer-PtNPs). All synthetic and assembling processes were stated in the Experiment Section. The TEM image in FIG. 10 shows the morphology of the synthesized PtNPs with a diameter of ˜4 nm. The assembled dual-nanoparticle-conjugated DNA-probe is preimmobilized in the sample recognition microwell by the magnetic field, which minimizes the complex chemical modification with the use of magnetism capturing. H₂O₂ and food dyes are also pre-injected into the amplification microwell and the indicator microwell (See FIG. S-3a), respectively. The MB Spinchip then becomes ready to use. In Step (i), by rotating the Spin unit, four parallel channels in the MB-SpinChip are rapidly connected with four sample recognition microwells, while keeping all exhaust outlets open. As such, the one-time sample injection allows the sample to be efficiently distributed into four sample recognition microwells (See FIG. 12B). In Step (ii), after sample introduction, all inlets and exhaust outlets are sealed by manually rotating the sectorial Spin unit to disconnect separate bar-char channels with the common inlet, thus forming multiple hermetical reaction chambers (See FIG. 12C). Meanwhile, the sample recognition is initiated by mixing with the preloaded aptasensor after the sample injection. The sample containing pathogenic microorganisms reacts with the immobilized aptasensor to activate the specific binding reaction between the pathogen and aptamer-PtNPs to form the binding complexes. Under the magnetic field effect, the unreacted aptasensor is retained at the bottom of the sample recognition microwell, whereas the binding complexes become free in the solution. In Step (iii), by holding the right end of the MB-SpinChip, the binding complexes with PtNPs in parallel sample recognition microwells are shaken down into catalytic amplification microwells to mix with the H₂O₂ solution (See FIG. 12D). Oxygen gas (O₂) is generated quickly from H₂O₂ under the catalysis of PtNPs, causing a dramatic pressure increase in the sealed parallel chambers without interference from other chambers because the “OFF” status of the Spin unit. Thus, the pressure cross interference problem in the multiplexed bar-chart chips is successfully addressed by the Spin unit. High pressure will be transduced to the visual multiplexed bar-chart signal by driving different food dyes to move in different bar-chart channels. Because more pathogens result in higher pressure as indicated by a longer color dye bar-chart signal, the pathogen concentration is proportional to the moving distance of the dyes, achieving visual quantitative detection of pathogens. Likewise, different aptasensors in different detection units can be simultaneously applied in a single MB-SpinChip for multiplexed pathogen detection with high throughput. In the absence of the pathogen, the specific aptamer-PtNPs will not come off from the magnetic beads and stay in the recognition microwells due to the magnetic attraction. Hence, no O₂ generation reaction by the catalyst of PtNPs happens, following without noticeable bar-chart movement.

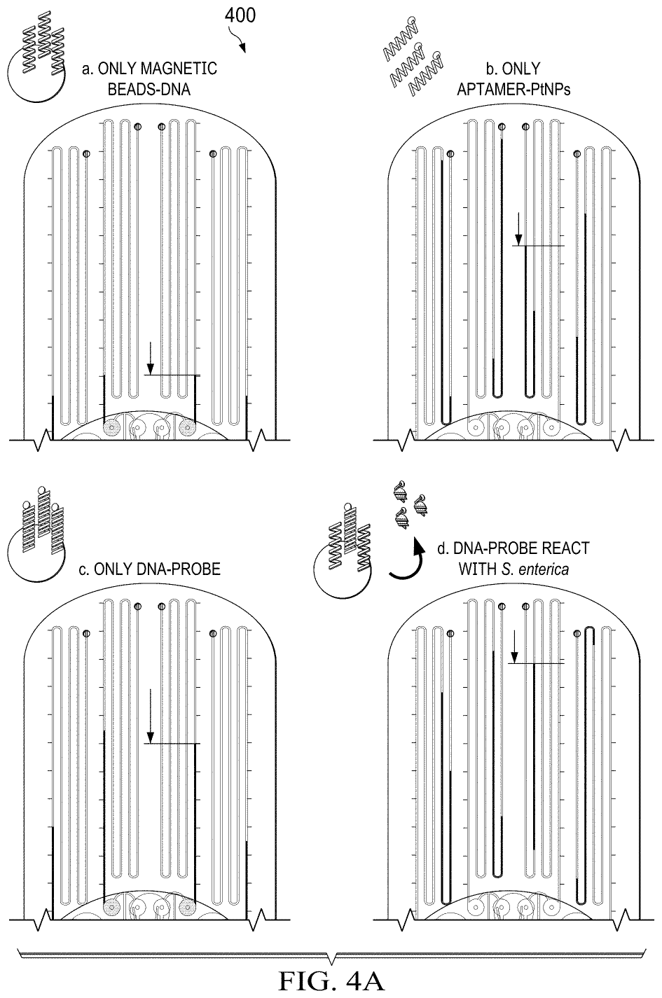

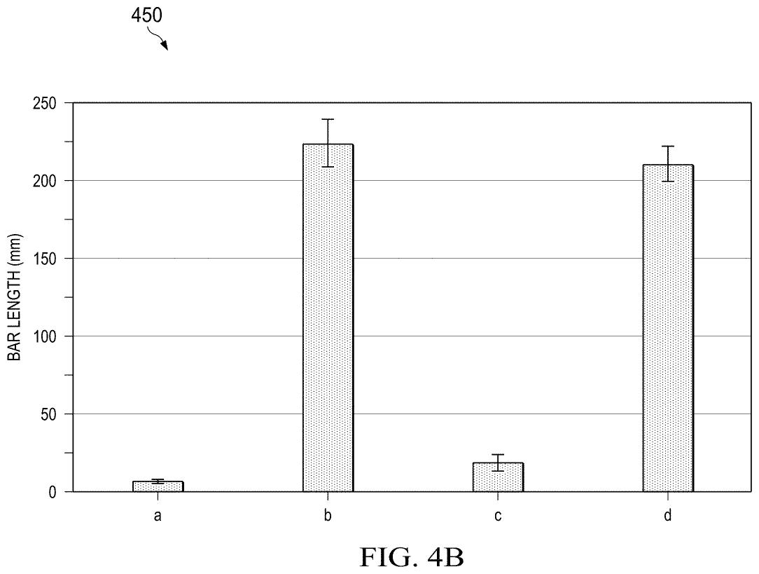

FIG. 4A shows a photograph 400 and corresponding histogram in FIG. 4B of visual bar-chart at different conditions. FIG. 4A(a) Only magnetic beads-DNA, FIG. 4A (b) Only PtNPs-aptamer, FIG. 4A (c) Only DNA-probe, and FIG. 4A (d) DNA-probe reacting with S. enterica. The blue arrows and dotted lines indicate the end point of the dye bar and the quantitative values of scale marker, respectively. The standard deviation was obtained from four parallel measurements.

We then conducted a series of experiments in the presence of different components of the aptasensor to demonstrate the feasibility of the proposed mechanism, while using S. enterica as the model pathogen. Four solutions with different DNA components were prepared for the visual bar-chart detection based on the MB-SpinChip. Solution (a) in the presence of only magnetic beads-DNA shows a negligible bar chart signal (FIG. 4A-a). Because a few Fe₃O₄-nanoparticle beads under the magnetic field were still shaken into the amplification microwell, a weak catalysis by Fe₃O₄-nanoparticle beads generated little O₂ and a short bar length. In the presence of only PtNPs-aptamer, a strong bar-chart signal of more than 200 mm was detected, due to the robust catalytic activity of PtNPs from the conjugated complex PtNPs-aptamer. However, when PtNPs-aptamer was hybridized on the magnetic beads forming the aptasensor probe, a much weaker bar-chart signal (less than 30 mm) was measured (FIG. 4A-c), which validated the magnetic capturing is effective. The DNA hybridization could immobilize most PtNPs-aptamers, but a few non-hybridized PtNPs-aptamer were free in the solution and reacted with H₂O₂ forming a short bar length, which could be minimized as the background signal by optimizations. Once we had all the necessary components for the bar-chart assays including a pathogen sample and its specific aptasensor, a significant increase in the bar length was obtained with a mean value of 210 mm. It is much greater than the background result without the pathogen (mean value, 19 mm), implying the specific recognition and detection of the pathogen from the MB-SpinChip with the aptasensor. Taken together, our results clearly demonstrated the feasibility of our MB-SpinChip that the nanoparticle mediated magnetic aptasensor can recognize and bind with the pathogen to trigger a pressure catalytic amplification for the visual quantitative pathogen detection.

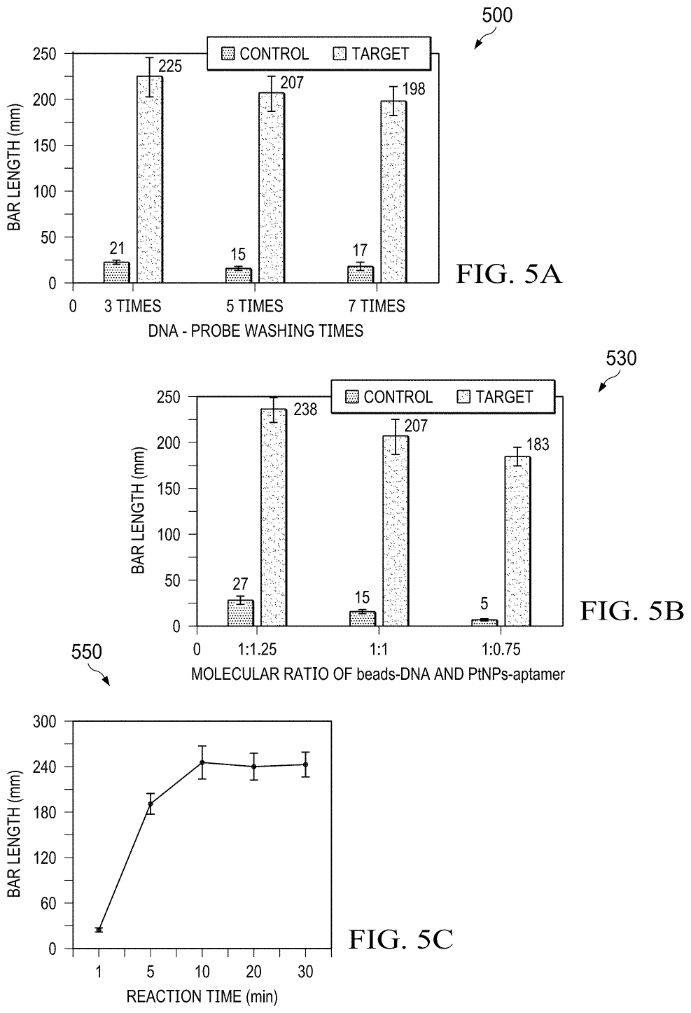

FIG. 5A shows optimization of a DNA probe washing times, FIG. 5B shows a concentration ratio of beads-DNA and PtNPs-aptamer, and FIG. 5C shows the reaction time between DNA probes and pathogens. The error bars represent standard deviations from four parallel measurements.

Next, the condition optimization and selectivity of the MB-SpinChip is discussed. Several parameters were optimized for longer bar length against the background signal, namely, DNA probe washing times, the ratio of beads-DNA and PtNPs-aptamer, and the reaction time. To minimize the amount of unhybridized PtNPs-aptamers, we first optimized the DNA probe washing times on the MB-SpinChip. FIG. 5A shows different bar lengths in the presence of the target, S. enterica. The bar length decreased slightly with the increase of washing times from 3 to 7. Considering the biggest absolute increment between the target and the control, three times was selected as the optimal DNA probe washing times. Besides, the molecular ratio of beads-DNA and PtNPs-aptamer was optimized for the maximization of the target response increment against the control signal. As seen in FIG. 5B, both the target and control signals increased with the increase of the ratios from 1:0.75 to 1:1.25 ([beads-DNA]: [PtNPs-aptamer]). The calculated bar length difference between the target and the control also increased with the increase of the ratio, reaching the maximum length at 1:1.25. Hence, the molecular ratio of 1:1.25 was chosen as the optimal molecular ratio of beads-DNA and PtNPs-aptamer. In addition, to ensure efficient binding between the DNA probe and the pathogen, the reaction time was optimized from 1 min to 30 min. The bar length increased with the increase of the reaction time from 1 min to 10 min, and then achieved a saturated level after 10 min (FIG. 4C). Herein, 10 min was selected as the optimal reaction time between the DNA probe and the pathogen.

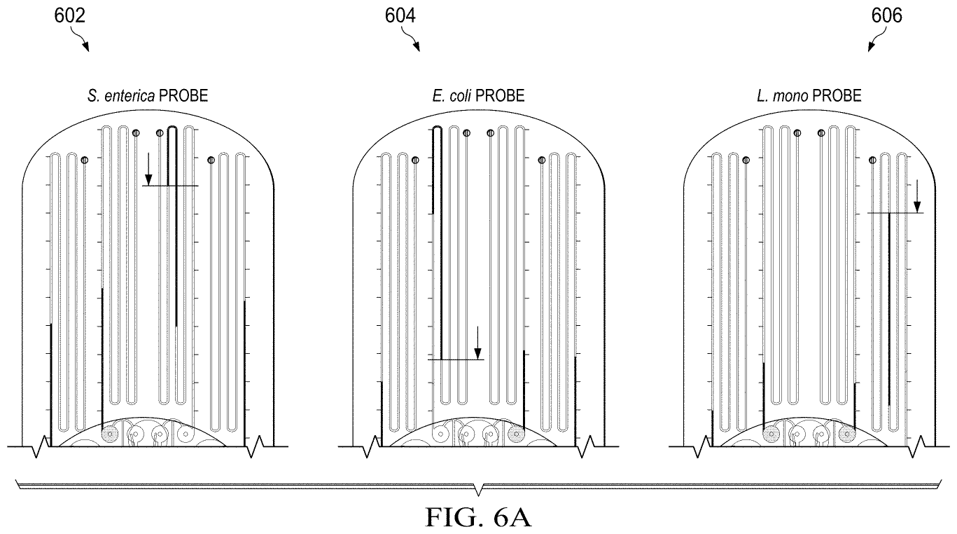

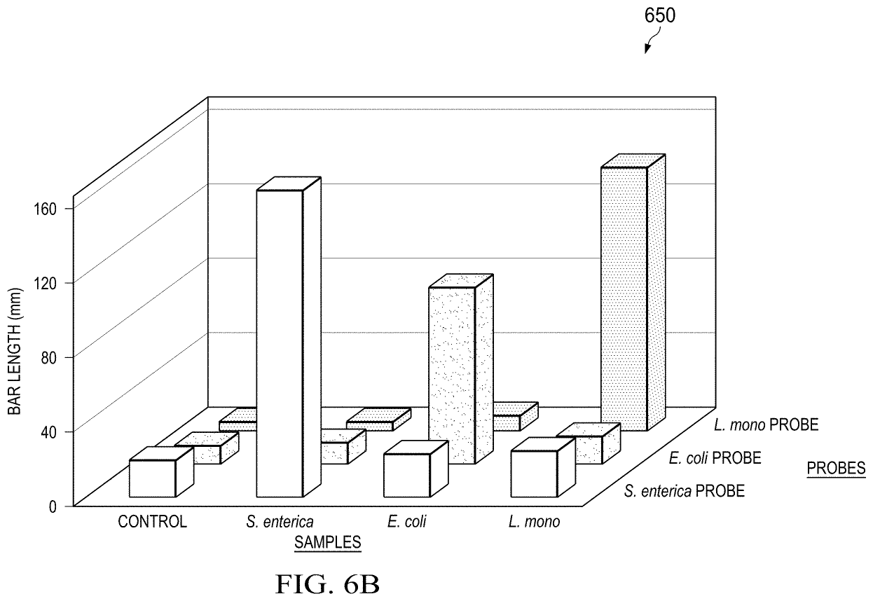

FIG. 6A shows selectivity investigation of an exemplary embodiment of the MB-SpinChip with different DNA probes for S. enterica 602, E. coli 604, and L. monocytogenes 606 (abbreviated as L. mono) by their corresponding photographs and bar-length histograms 650 in FIG. 6B.

Considering the application of the MB-SpinChip in complex biological matrixes, the selectivity of three types of aptasensors targeting S. enterica, E. coli, and L. monocytogenes was evaluated using the MB-SpinChip. As shown in FIG. 6A, the S. enterica aptamer-probe was used for the detection of 400 CFU/mL S. enterica and other pathogens at higher concentrations (more than 10³ folds). Only the specific pathogen, the S. enterica sample, showed a long bar-chart signal of 162 mm, whereas the other two samples including E. coli and L. monocytogenes only showed very weak bar-chart signals of 30 mm, which was almost the same as the control experiment. Similarly, the E. coli aptasensor and L. monocytogenes aptasensor were also investigated with different pathogens. The 10⁶ CFU/mL E. coli and 10⁶ CFU/mL L. monocytogenes gave barchart readings of 94 mm and 140 mm (See FIG. S-4 in detail), respectively. On the contrary, the non-specific pathogen samples at a higher concentration only showed a bar length of less than 20 mm. Therefore, the result confirmed the high specificity of our MB-SpinChip.

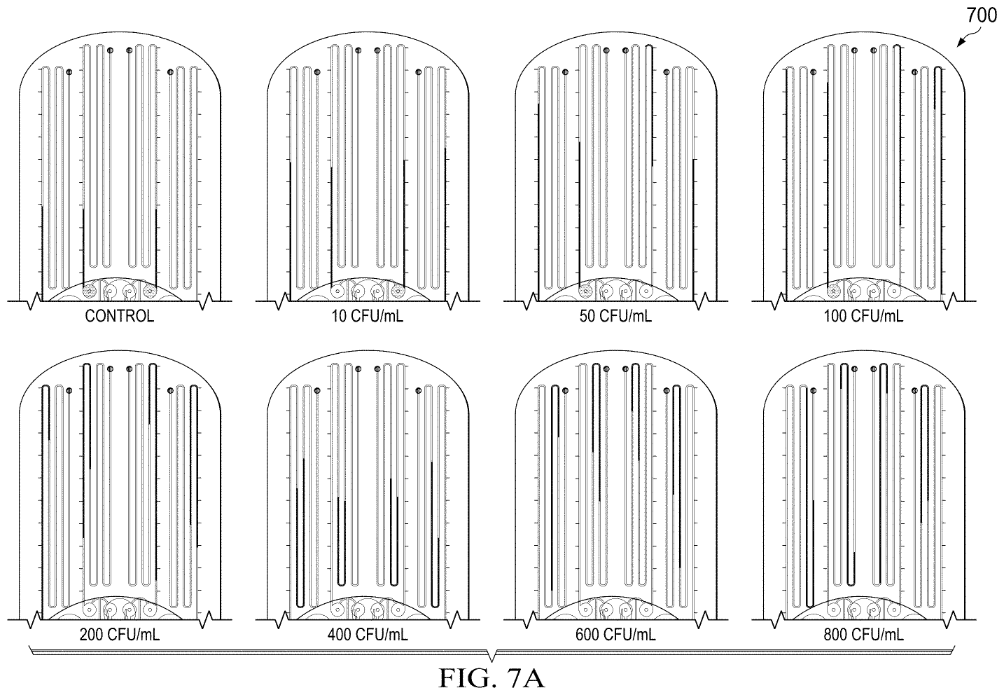

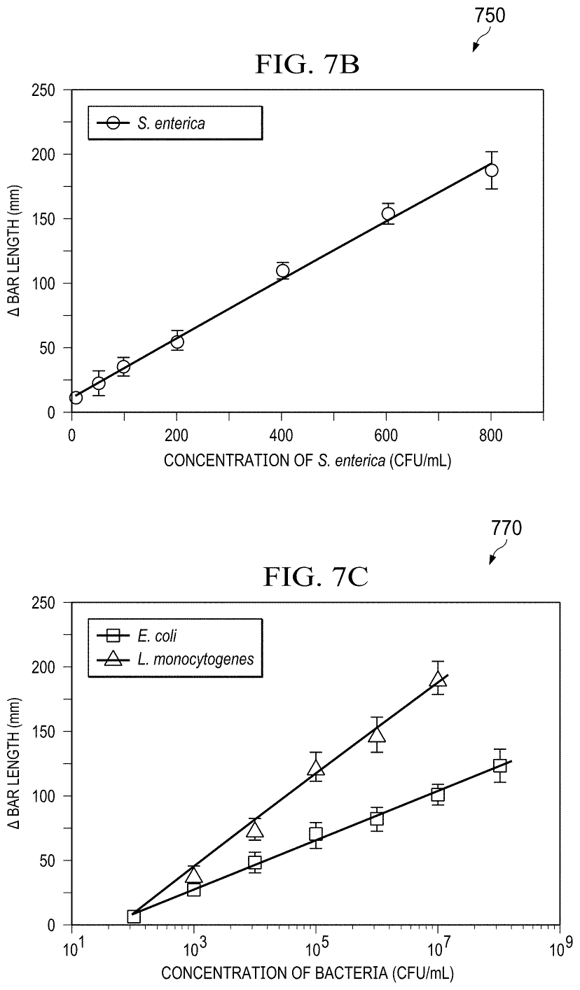

FIG. 7A shows an exemplary embodiment of visual quantitative pathogen detection 700 using the MB-SpinChip with photographs of visual bar-chart responses to different concentrations of S. enterica from 10 CFU/mL-800 CFU/mL. The arrows and dotted lines indicate the end point of the dye bars corresponding to different values on the scale bar. FIG. 7B shows a calibration curve 750 of the bar-chart signal versus different concentrations of S. enterica (green line). FIG. 7C shows calibration curves 770 of the bar-chart signal versus different concentrations of E. coli from 102 CFU/mL˜108 CFU/mL (blue line) and L. monocytogenes 102 CFU/mL˜107 CFU/mL (red line). The error bars represent standard deviations from four parallel measurements.

Visual Quantitative Detection of Pathogens. After optimization, the MB-SpinChip was first applied to visual quantitative detection of individual pathogens, S. enterica, E. coli, and L. monocytogenes. S. enterica at various concentrations, with four parallel measurements using the MB-SpinChip. As shown in FIG. 7A, the visual bar chart signal increased with the increase of the concentration of S. enterica from 0 CFU/mL to 800 CFU/mL. Taking the length of the 0 CFU/mL of S. enterica as the blank, the Δlength between the target to the blank lengths of different concentrations of S. enterica was calculated and plotted versus the concentration. An excellent linear relationship between the mean □length and the S. enterica concentration was obtained in the range of 10 CFU/mL˜800 CFU/mL with the R² value of 0.994 (FIG. 7B). The limit of detection (LOD) of 6.74 CFU/mL S. enterica was achieved based on 3 folds of standard deviation (SD) above the blank value. Compared with other POCTs for the detection of S. enterica, our instrument-free method has higher detection sensitivity than the SERS-based lateral flow strip (LOD, 27 CFU/mL) and the colorimetric method (LOD, 100 CFU/mL) using the UV-vis absorption spectrum. The sensitivity of our method is even comparable to that of DNA amplification-based lateral flow devices with LOD of 4 CFU/mL.⁶⁸

Following a similar protocol, different concentrations of E. coli and L. monocytogenes were separately tested by their corresponding aptasensors on the MB-SpinChip and their absolute bar-chat differences were plotted in FIG. 6C. It can be seen that the calibration curves for E. coli and L. monocytogenes were linearly fitted in the range from 10² CFU/mL˜10⁸ CFU/mL (R²=0.996) and 10² CFU/mL˜10⁷ CFU/mL (R²=0.995), respectively. The LOD values of E. coli and L. monocytogenes are 16 CFU/mL and 20 CFU/mL, respectively. Even comparing with other DNA amplification methods, the sensitivity of our method is better than the LOD of 100 CFU/mL for E. coli by the LAMP with Electrochemical Impedance method⁶⁹ and comparable to the LOD of 10 CFU/mL for L. monocytogenes by the polymerase chain reaction (PCR) with electrochemiluminescence-based gene-sensing.⁷⁰ These results indicate high detection sensitivities of our bar-chart SpinChip for visual quantitative detection of multiple pathogens, and laid a solid foundation for us to explore its capacity in the subsequent multiplexed pathogen detection.

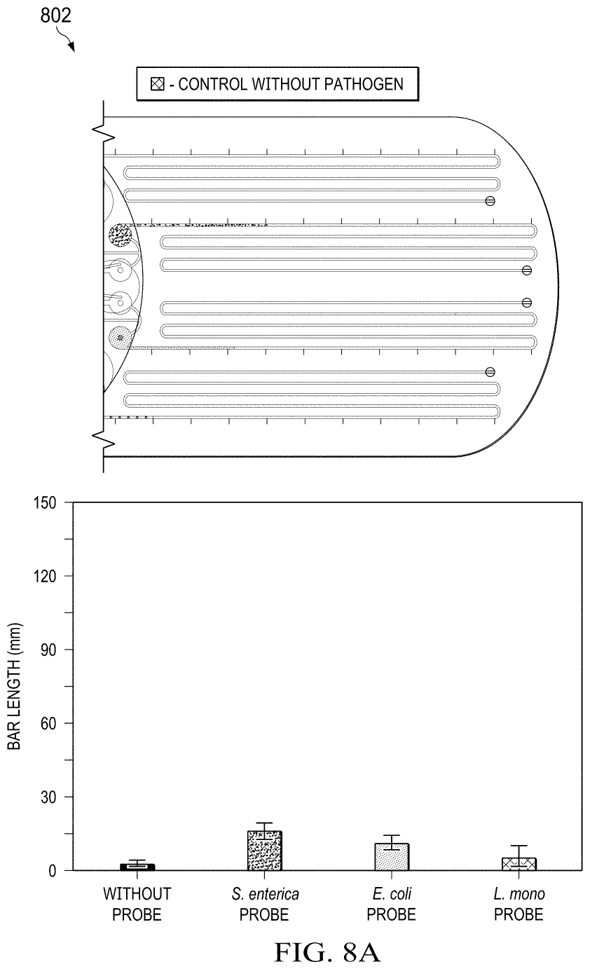

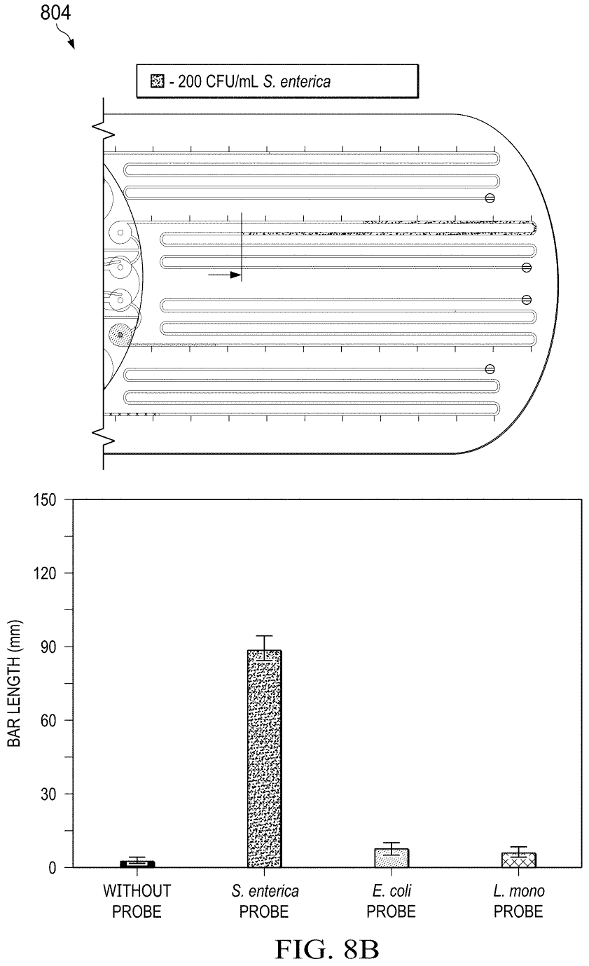

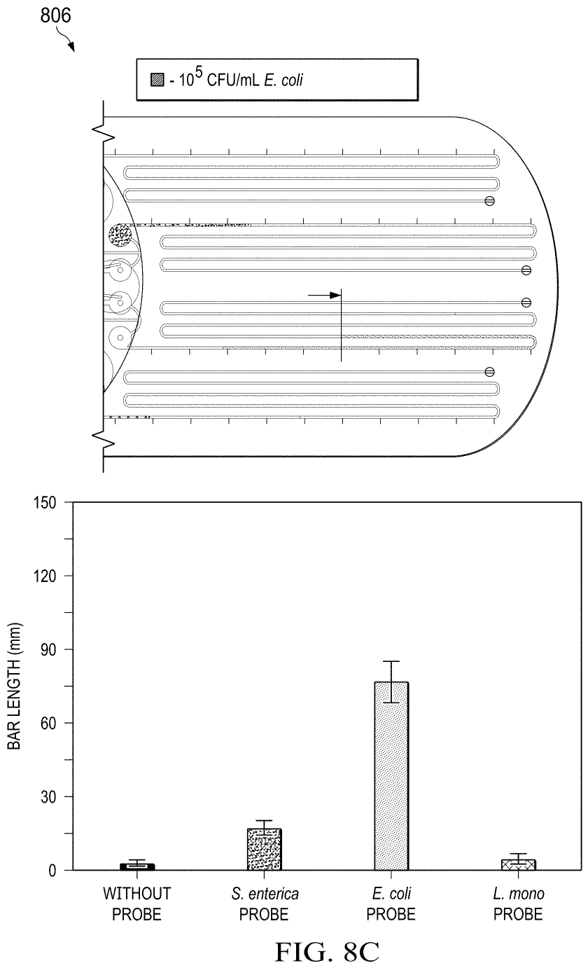

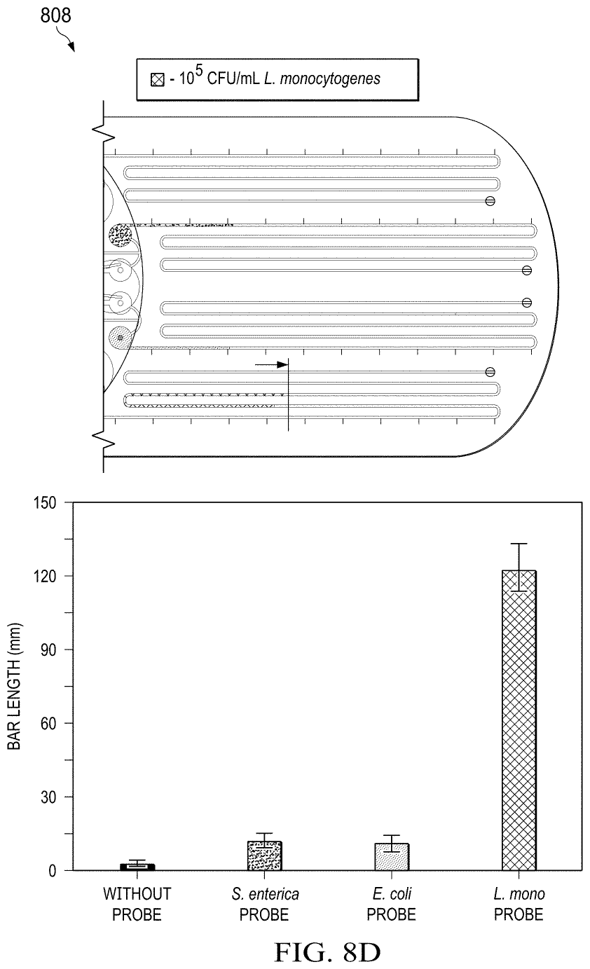

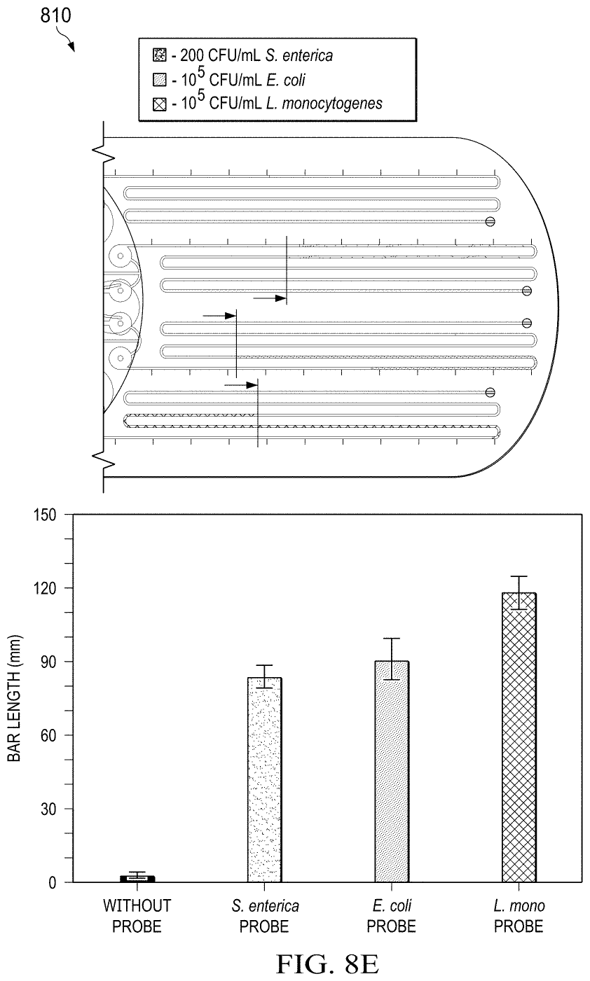

FIGS. 8A-8E shows an exemplary embodiment of multiplexed pathogen detection. FIGS. 8A shows a control without pathogens on the MS-SpinChip 802. FIGS. 8A-8D shows testing individual pathogens using the MS-SpinChip. FIG. 8B shows 200 CFU/mL S. enterica 804, FIG. 8C shows 105 CFU/mL E. coli 806, and FIG. 8D shows 105 CFU/mL L. monocytogenes (abbreviated as L. mono) 808. FIG. 8E shows simultaneous detection of three types of pathogens on a single MB-SpinChip 810. The pathogen concentrations in FIG. 8E correspond to the same concentrations in FIG. 8B-8D, respectively. The various bars represent the control signal, S. enterica signal, E. coli signal, and L. mono signal, respectively.

As multiple pathogens co-exist, multiplexed detection becomes increasingly important, especially in testing complex biological samples and unknown samples.⁷¹⁻⁷⁴ The multiplexed measurement can not only enhance the throughput and convenience for higher detection efficiency, but also provide richer information at lower cost from a single assay.³⁰ Since the Spin unit solved a major issue in multiplexed bar-chart microfluidics, multiple aptasensors were simultaneously integrated in one MB-SpinChip for multiplexed detection of pathogens. Herein, S. enterica, E. coli, and L. monocytogenes were chosen as a complex model of foodborne diseases. We first injected different aptamer-probes into different sample recognition microwells and four different food dyes into four indicator microwells to distinguish different targets. Then, after three different aptasensors were integrated on the same bar-chart chips, the multiplexed SpinChips were first used to test individual targets. As shown in FIGS. 8A-8E, sample-A without any pathogens (the negative control) was measured and only showed weak background bar chart signals. Because the concentrations of different aptamer-probes were optimized for higher sensitivity to corresponding pathogens, slightly different background signals were observed when testing the same mixture using different aptasensors. However, all the negative control signals were below 20 mm. But when sample-B including 200 CFU/mL S. enterica was detected using the MB-SpinChip integrated with three aptasensors, a significant increase in green bar to 89 mm while no other color bars was observed, as indicated by the bar-chart graph in FIG. 7B. Similarly, Sample-C including 105 CFU/mL E. coli and Sample-D including 105 CFU/mL L. monocytogenes were separately tested by using different MB-SpinChips. FIGS. 7C and 7D indicated dramatic bar-chart signal increases of 77 mm in the red bar (i.e. E. coli) and 123 mm in the blue bar (L. monocytogenes), with no noticeable increases of other color bars. These results confirmed that individual pathogens could be effectively and quantitatively detected by MB-SpinChip, when different aptasensors were integrated on the same bar-chart chips.

The multiplexed detection capacity was further tested by simultaneously detecting three types of pathogens that co-existed in one sample using our multiplexed bar-chart SpinChip. As shown in FIG. 7E, when 200 CFU/mL S. enterica, 105 CFU/mL E. coli, and 105 CFU/mL L. monocytogenes from a single injection were detected on the same chip, their corresponding bar-chart channels showed strong signals, i.e. a 84 mm green bar, a 91 mm red bar, and a 118 mm blue bar, respectively. Their bar lengths of Sample-E in the simultaneous detection are consistent with their corresponding values tested in Sample-B, C, D in the presence of only one pathogen in each sample. Hence, this further confirmed the strong multiplexing capacity of our MB-SpinChip in the visual quantitative detection of multiple pathogens simultaneously, with-out the aid of any equipment. To validate our MB-SpinChip in multiplexed detection, a food sample, apple juice, spiked with S. enterica, E. coli, and L. monocytogenes were simultaneously measured using our MB-SpinChips b. All the recovery values from different pathogens were determined at the satisfactory level between 95%˜110%. And all the coefficient variations are less than 10%. Consequently, the aptasensor-integrated MB-SpinChip can be effectively applied for the multiplexed detection of pathogens in food samples.

In summary, we have developed a portable, low-cost and instrument-free multiplexed bar-chart SpinChip integrated with PtNPs-mediated magnetic aptasensor for the visual quantitative and simultaneous detection of multiple pathogens. We used S. enterica as a model to develop the MB-SpinChip, and then successfully extended to the multiplexed detection of three pathogens, S. enterica, E. coli, and L. monocytogenes, in which the newly developed Spin unit played a crucial role in the multiplexed bar-chart chip. Three major types of foodborne pathogens were quantified simultaneously using the MB-SpinChip with high detection sensitivity. LODs of about 10 CFU/mL were readily achieved, without using any equipment. Additionally, compared to other glass or glass/polymer based bar-chart V-chips, our multiplexed bar-chart SpinChip does not (1) need sophisticated operation procedures, and (2) complicated and costly photolithography and chemical etching in other bar-chart chip fabrication; (3) The PMMA substrate allows lower-cost and more environment-friendly bioassays, compared to glass-based bar-chart chips; (4) Nanoparticle-mediated catalysis is not as sensitive to ambient temperatures as enzymes which were commonly used in other bar-chat chips.

Multiple important features of the MB-SpinChip are appealing as a universal POC platform for the multiplexed detection of pathogens and other biochemicals. (i) The visual quantitative detection can be achieved without using any specialized instrument. Instead of relying on complicated pneumatic pumps and expensive signal detectors, PtNPs-mediated catalytic pressure amplification integrated on the MB-SpinChip provides robust driving force to transduce the pressure signal into visual dye bar charts. A user-friendly quantitative barchart readout can be conducted on the MB-SpinChip similarly to a traditional thermometer. (ii) Multiplexed detection of multiple pathogens can be accomplished from a single assay. By integrating the innovative Spin unit on the MB-SpinChip, we can readily deliver reagents and samples from one inlet to different channels without causing pressure cross interference problems during the subsequent detection step. Integrated with multifarious aptasensors, simultaneous measurements of multiple pathogens can be efficiently completed on a single MB SpinChip at a time. (iii) The method owns high simplicity. Our method utilizes specific aptasensors to recognize bacterial microorganisms directly, without the need of cell lysis and other complicated sample preparation procedures. (iv) The PtNPs-mediated magnetic aptasensor-integrated MB-SpinChip has great potential and wide applications in the POC detection of a wide range of pathogens and biochemicals in food safety, environment surveillance, and infectious disease diagnosis at the point of care and other low-resource settings.

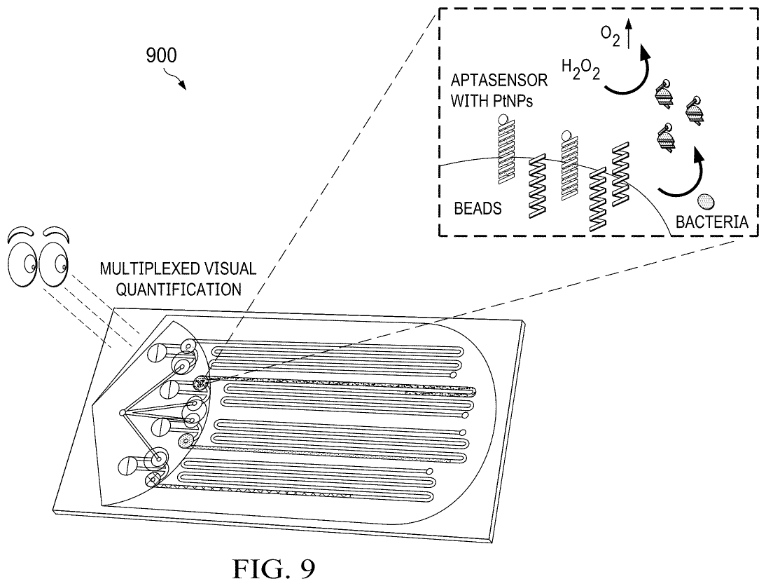

FIG. 9 shows an exemplary embodiment of multiplexed visual quantification. In an illustrative embodiment, The DNA biosensor was composed of two functional DNA probes, beads-DNA for the immobilization of DNA biosensor and PtNPs-aptamer for the specific pathogen recognition and nanoparticle-mediated pressure amplification. First, cDNA-NH₂ containing the same DNA sequence as the aptamer was conjugated with the carboxyl magnetic beads (beads-COOH). 1 mL 0.25 mg/mL of carboxyl beads were conjugated with 200 nM cDNA-NH₂ through the crosslink of 12.5 μg/mL EDC.HCl and 15 μg/mL SulfoNHS. The mixture solution was adjusted to pH 8.0 by NaHCO₃ and kept shaking for 3 hours at room temperature. The conjugated beads-DNA was centrifuged (10000 rpm, 5 min), washed with a 1× PBS buffer for 3 times, and finally solved in 1× PBS as the 100 nM stock solution of beads-DNA. For the PtNPs-aptamer, the aptamer was synthesized with thiol-modification for the chemical conjugation with the PtNPs (See FIG. 10) which were synthesized according to the protocol from a previously published report.¹ TEM was used to characterize their morphology using a Transmission Electron Microscope (TEM) from JEOL Ltd (Peabody, Mass.). 1 μM thiol-aptamer (50 μL) was activated with 0.1 mM Tris (2-carboxyethyl) phosphine hydrochloride (TCEP) by shaking 1 hour at room temperature. The prepared fresh PtNPs (200 μL, 25 nM) and 2× PBS buffer (250 μL) was added into the activated thiolaptamer solution and kept shaking for 24 hours in darkness. The conjugated PtNPs-aptamer was centrifuged at 10000 rpm for 5 min, then washed with 1× PBS buffer for 3 times, and finally dissolved in 1× PBS (500 μL) as the 100 nM stock solution of PtNPsaptamer. Further, 500 μL 100 nM of beads-DNA and the corresponding PtNPs-aptamer (100 nM) were mixed and centrifuged (10000 rpm, 5 min) to remove the supernatant, and finally dissolved in a 1 mL binding buffer. The hybridization reaction was incubated at 37° C. for 4 hours to obtain the DNA biosensor. The hybridized DNA biosensor was centrifuged at 10000 rpm for 5 min, and washed with 1× PBS buffer for 3 times, before it was dissolved in 1× PBS (500 μL) as the 100 nM stock solution of the DNA biosensor.

Turning now to the assay procedure on the MB-SpinChip, Different concentrations of samples were prepared from 10˜800 CFU/mL for S. enterica, 10²˜10⁸ CFU/mL for E. coli, 10²˜10⁷ CFU/mL for L. monocytogenes with the final volume of 40 μL. First, the top Layer-1 was spun to locate the exhaust outlets of Layer-1 and Layer-2 and connect the four branched channels of Layer-1 to four sample inlets of Layer-2 (See FIG. 12B). The sample solution was injected from the inlet of Layer-1 and distributed into four sample recognition microwells to react with the preloaded DNA biosensor. Then, the top Layer-1 was spun with a certain angle to seal all sample inlets and exhaust outlets and formed a hermetic reaction chamber by the clamp (See FIG. S-3c). The sample was incubated with DNA biosensors for the specific recognizing reaction for 10 min at room temperature. Through the direction from the sample recognition microwell to the amplification microwell, the sample solution including the PtNPs reporter was shaken into the amplification microwell and mixed with the preloaded H₂O₂ (See FIG. 12D). A moderate oxygen-producing reaction was initialized by the PtNPs catalyst in the presence of H₂O₂ and increased the pressure of the sealed reaction chamber. Under the increasing pressure, the food dye was pushed into the bar-chart channel and generated a visual bar chart with a quantitative length. Finally, the mean length of four bar charts after 10 min reaction was calculated and recorded as the readout result.

FIG. 10 shows a TEM photograph 1000 of PtNPs.

FIGS. 11A-11E show a schematic of an exemplary embodiment of the MB-SpinChip with a Layout design of different layers. FIG. 11F shows a 3D schematic 1160 of the Spin-unit section.

FIGS. 12A-12D shows an exemplary embodiment of operation steps of an exemplary embodiment of the MB-SpinChip, including (a) Standby condition: preloading H₂O₂ reagent 1206 and Food dye 1204 onto an MB SpinChip 1200 having magnets 1202, (b) Connect and inject: opening exhaust outlet and connecting the branched channels with sample inlets 1208 for sample injection, (c) Spin and seal: spinning 1210 the Spin-unit to seal 1212 the exhaust outlets and sample inlets, (d) Shake and read: shaking down the samples into the substrate microwells 1216 to activate the O₂ generation with the bar-chart readout.

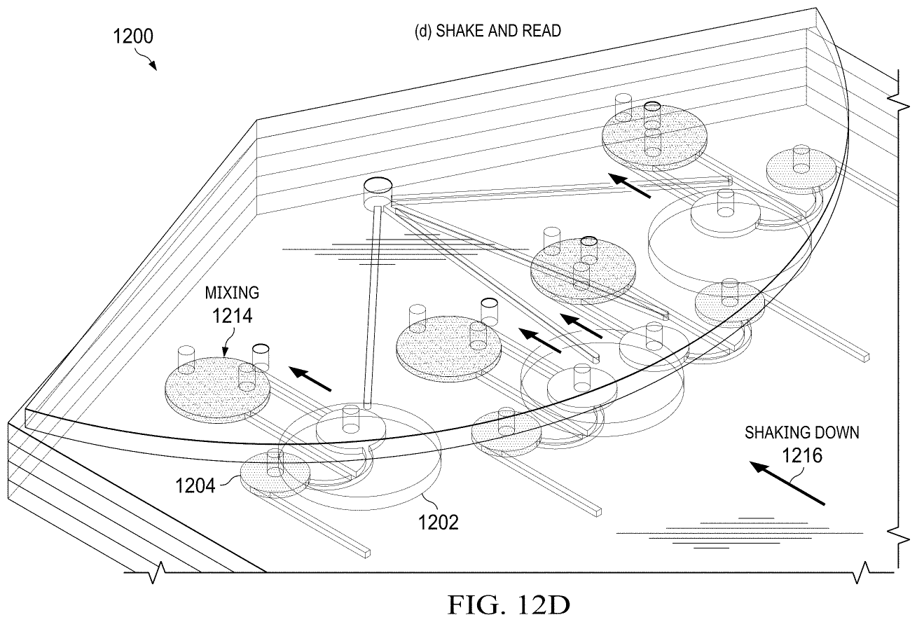

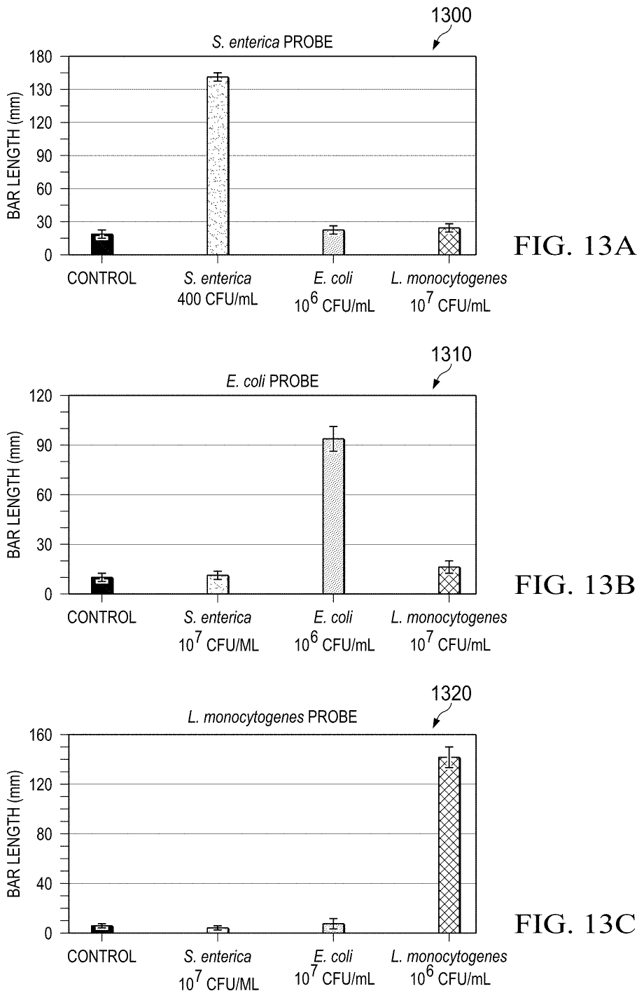

FIGS. 13A-13C show selectivity investigation of the MB-SpinChip with different DNA probes for FIG. 13A S. enterica 1300, FIG. 13B E. coli 1310, and FIG. 13C L. monocytogenes 1320. The standard deviation was obtained from four parallel measurements.

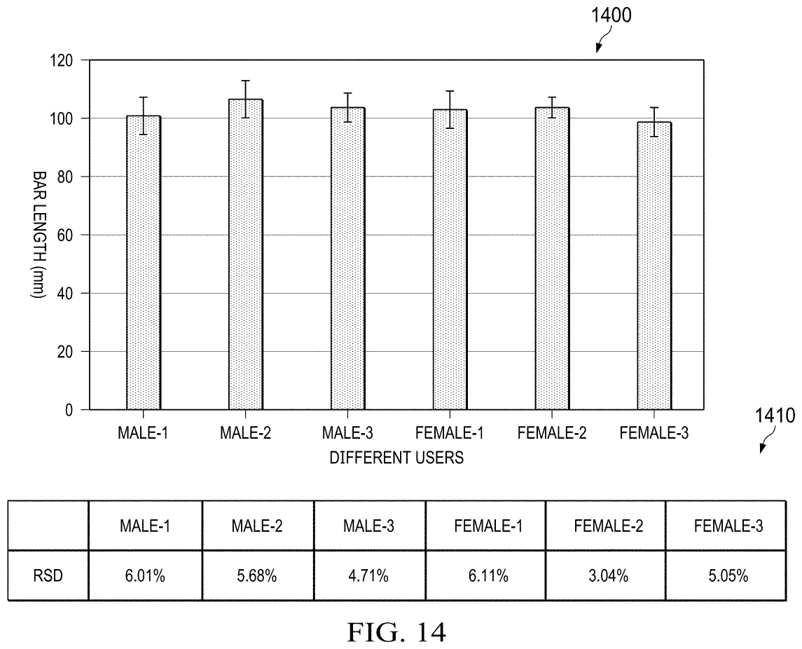

FIG. 14 shows a response 1400 of an exemplary embodiment of a MB-SpinChip operated by multiple users to 20 pM PtNPs and 30% H₂O₂ in 5 min. The relative standard deviations (RSD) 1410 were obtained from four parallel measurements in one assay.

It will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

What is claimed is:

1. A point-of-care testing (POCT) device for quantitative pathogen detection, comprising:
an inlet microwell for receiving a substance, the inlet microwell connected to a distribution channel to distribute the substance to an analyzing component;
the analyzing component comprising a first pathogen detection component, the first pathogen detection component comprising a platinum nanoparticle-labeled magnetic DNA-probe configured to recognize a first pathogen and then generate a fragmentary DNA-platinum nanoparticles, wherein the fragmentary DNA-platinum nanoparticles are configured to react with a substrate to generate gas to propel a dye through a bar-chart channel when a pathogen in the substance reacts with the platinum nanoparticle-labeled magnetic DNA-probe, wherein the platinum nanoparticle-labeled magnetic DNA-probe is configured to react with the first pathogen; and
at least one magnet configured to keep unreacted platinum nanoparticle-labeled magnetic DNA-probe in an amplification microwell, thereby inhibiting propulsion of the dye into the bar-chart channel when the pathogen is not detected.

2. The device of claim 1, wherein the distribution channel comprises a plurality of distribution channels and the analyzing component comprises a plurality of pathogen detection components, wherein each of the pathogen detection component is configured to detect a different pathogen, and wherein each of the pathogen detection components comprises the platinum nanoparticle-labeled magnetic DNA-probe configured to propel the dye through the bar-chart channel when the pathogen in the substance reacts with the platinum nanoparticle-labeled magnetic DNA-probe, wherein each of the platinum nanoparticle-labeled magnetic DNA-probes is configured to react with a different pathogen.

3. The device of claim 2, wherein each of the plurality of pathogen detection components is configured to receive a portion of a single sample received at the inlet microwell by rotating a spin unit (Status “ON”) to connect a common inlet to all bar-chart detection units.

4. The device of claim 2, wherein each of the plurality of pathogen detection components is isolated from the other ones of the plurality of pathogen detection components by rotating a spin unit (Status “OFF”) to disconnect a common inlet from the bar-chart channels such that a reaction to the pathogen in a first pathogen detection component does not propel the dye in a second pathogen detection component into the bar-chart channel corresponding to the second pathogen detection component.

5. The device of claim 2, wherein the at least one magnet comprises a magnet for each of a plurality of analyzing components.

6. The device of claim 1, wherein the platinum nanoparticle-labeled magnetic DNA-probe comprises a DNA hybridization between magnetic beads-complementary DNA and aptamer DNA-platinum nanoparticles.

7. The device of claim 1, further comprising:
a spin unit.

8. A multiplexed bar-chart spinchip for instrument free visual quantitative detection of multiple pathogens for point-of-care testing (POCT), comprising:
a layer-one sheet comprising a layer-one first surface and a layer-one second surface, the layer-one sheet further comprising a layer-one inlet connected to a plurality of layer-one branched channels and a plurality of layer-one exhaust outlets;
a layer-two sheet comprising a layer-two first surface and a layer-two second surface, the layer-two first surface in contact with the layer-one second surface, the layer-two sheet further comprising a plurality of layer-two sample inlets, a plurality of layer-two substrate inlets, a plurality of layer-two indicator inlets, a plurality of layer-two exhaust outlets, and a plurality of layer-two outlets;
a layer-three sheet comprising a layer-three first surface and a layer-three second surface, the layer-three first surface bonded to the layer-two second surface, a first surface of layer-three sheet further comprising a plurality of sample recognition microwells, a plurality of catalytic amplification microwells, a plurality of indicator microwells, a plurality of “T”-phase exchange channels, a plurality of connection channels, and a plurality of bar-chart channels, wherein each of the sample recognition microwells comprises a platinum nanoparticle-labeled magnetic DNA-probe, wherein the platinum nanoparticle-labeled magnetic DNA-probe in one of the sample recognition microwells is different from at least one of the other platinum nanoparticle-labeled magnetic DNA-probes in another one of the sample recognition microwells, wherein each of the sample recognition microwells is connected with a corresponding one of the catalytic amplification microwells and a corresponding one of the indicator microwells by a corresponding one of the “T”-phase exchange channels, wherein each of the connection channels is configured to specially connect the corresponding one of the sample recognition microwells and the corresponding one of the catalytic amplification microwells, wherein the plurality of sample recognition microwells, the plurality of catalytic amplification microwells, the plurality of indicator microwells, the plurality of “T”-phase exchange channels, the plurality of connection channels, and the plurality of bar-chart channels form a plurality of parallel microfluidic units;
a layer-four sheet comprising a layer-four first surface and a layer-four second surface, the layer-four first surface contacting the layer-three second surface, the layer-four second surface comprising a plurality of hollow microwells;
a plurality of magnets, each of the magnets residing in one of the hollow microwells; and
a layer-five sheet comprising a layer-five first surface and a layer-five second surface, the layer-five first surface contacting the layer-four second surface thereby securing the magnets in the hollow microwells;
wherein the magnets keep unreacted platinum nanoparticle-labeled magnetic DNA-probe in a corresponding one of the sample recognition microwells; and
wherein a dye is forced through one of the bar-chart channels when a sample is introduced with a pathogen corresponding to a particular platinum nanoparticle-labeled magnetic DNA-probe.

9. The multiplexed bar-chart spinchip of claim 8, wherein the platinum nanoparticle-labeled magnetic DNA-probes each comprise a DNA hybridization between magnetic beads-complementary DNA and aptamer DNA-platinum nanoparticles.

10. The multiplexed bar-chart spinchip of claim 8, wherein a distance that the dye moves is proportional to an analyte concentration, thus enabling quantitative detection of pathogens, without the aid of any detectors.

11. The multiplexed bar-chart spinchip of claim 8, wherein multiple different types of pathogens are quantitatively detected simultaneously from a single assay.

12. A point-of-care testing (POCT) device for quantitative pathogen detection, comprising:
an inlet microwell for receiving a substance;
an analyzing component configured to propel a dye through a bar-chart channel when a pathogen in the substance reacts with a catalyst-mediated magnetic DNA-probe; and
at least one magnet configured to keep unreacted catalyst-mediated magnetic DNA-probe in an amplification microwell, thereby inhibiting propulsion of the dye into the bar-chart channel when the pathogen is not detected;
wherein the inlet microwell is connected to a distribution channel to distribute the substance to the analyzing component.

13. The POCT device of claim 12, further comprising a catalyst nanoparticle-mediated magnetic DNA-probe, wherein the catalyst nanoparticle-mediated magnetic DNA-probe is configured to recognize the pathogen and generate fragmentary DNA-catalyst nanoparticles.

14. The POCT device of claim 13, wherein the fragmentary DNA-catalyst nanoparticles are configured to react with a substrate to generate gas to propel the dye through the bar-chart channel when the pathogen in the substance reacts with the catalyst nanoparticle-labeled magnetic DNA-probe.

15. The POCT device of claim 12, wherein the catalyst nanoparticle-labeled magnetic DNA-probe is configured to react with a first pathogen, and wherein the catalyst nanoparticle-labeled magnetic DNA-probe is configured to not react with a second pathogen.

16. The POCT device of claim 12, wherein the catalyst comprises platinum.

17. A method of fabricating a point-of-care testing (POCT) device for quantitative pathogen detection, the method comprising:
forming an inlet microwell for receiving a substance, the inlet microwell connected to a distribution channel to distribute the substance to an analyzing component;
forming the analyzing component comprising a first pathogen detection component, the first pathogen detection component comprising a platinum nanoparticle-labeled magnetic DNA-probe configured to recognize a first pathogen and then generate a fragmentary DNA-platinum nanoparticles, wherein the fragmentary DNA-platinum nanoparticles are configured to react with a substrate to generate gas to propel a dye through a bar-chart channel when a pathogen in the substance reacts with platinum nanoparticle-labeled magnetic DNA-probe, wherein the platinum nanoparticle-labeled magnetic DNA-probe is configured to react with the first pathogen; and
forming a layer comprising at least one magnet configured to keep unreacted platinum nanoparticle-labeled magnetic DNA-probe in an amplification microwell, thereby inhibiting propulsion of the dye into the bar-chart channel when the pathogen is not detected.
